(19) United States
(12) Patent Application Publication
(10) Pub. No.: US 2013/0210899 A1
(43) Pub. Date: Aug. 15, 2013

(54) Method and Therapeutic for the Treatment and Regulation of Memory Formation

(71) Applicant: Marcelo Andres Wood, Irvine, CA (US)

(72) Inventor: Marcelo Andres Wood, Irvine, CA (US)

(73) Assignee: The Regents of the University of California, Oakland, CA (US)

(21) Appl. No.: 13/810,156

(22) PCT Filed: Jul. 28, 2011
(86) PCT No.: PCT/US2011/045790

§ 371 Date: Apr. 30, 2013

Related U.S. Application Data
(60) Provisional application No. 61/369,552, filed on Jul. 30, 2010.

(51) Int. Cl. A61K 48/00; A61K 31/165; A61K 31/415

(52) U.S. Cl. CPC A61K 48/0066; A61K 31/415; A61K 31/165
USPC 514/44 R; 514/406; 514/619; 548/373.1; 564/163

(57) ABSTRACT

A methodology and pharmaceutical and gene therapies for the treatment and regulation of memory function are provided. The invention identifies specific HDAC, and in particular, HDAC3 and HDAC4 as negative regulators of memory formation and specifically targets one or both HDAC3 and HDAC4 for down-regulation. By specifically targeting HDAC3 and HDAC4 with small molecule inhibitors and gene therapies it is possible to provide a powerful therapeutic approach to facilitate gene expression during memory formation that can lead to the regulation and treatment of memory disorders.

### STATEMENT OF FEDERAL SUPPORT

The U.S. Government has certain rights in this invention pursuant to Grant Nos. Grant No(s). Ro1MH081004 awarded by the National Institute of Mental Health and R01DA025922 awarded by the National Institute on Drug Abuse, and to NRSA Fellowship F31 MH85494 awarded by the National Institute of Health.

### FIELD OF THE INVENTION

The current invention is directed to methods and therapeutics for use in regulating memory function.

### BACKGROUND OF THE INVENTION

Transcription is thought to be a key step for long-term memory processes. (See, e.g., Alberini, Physiol Rev 89:121-145, 2009, the disclosure of which is incorporated herein by reference) Transcription is promoted by specific chromatin modifications, such as histone acetylation, which modulate histone-DNA interactions. (Kouzarides et al., Cell 128:693-705, 2007, the disclosure of which is incorporated herein by reference.) Modifying enzymes, such as histone acetyltransferases (HATs) and histone deacetylases (HDACs), regulate the state of acetylation on histone tails. In general, histone acetylation promotes gene expression, whereas histone deacetylation leads to gene silencing. Numerous studies have shown that a potent HAT, cyclicAMP response element binding protein (CREB)-binding protein (CBP), is necessary for long-lasting forms of synaptic plasticity and long-term memory. (For a review see, Barrett and Wood, Learn Mem 15:460-467, 2008, the disclosure of which is incorporated herein by reference.) Likewise, mouse models with a loss of CBP's HAT function all show attenuated histone acetylation and impaired long-term memory formation. (See, Alarcon, J. M., et al., Neuron, 42, 947-959, 2004; Barrett, R. M., et al., Neuropsychopharmacology, (in press); Bourtchouladze, R., et al. Proceedings of the National Academy of Sciences USA, 100, 10518-10522, 2003; Korzus, E., et al., Neuron, 42, 961-972, 2004; Oike, Y., et al. Human Molecular Genetics, 8, 387-396, 1999; Stefanko, D. P., et al., Proceedings of the National Academy of Sciences USA, 106, 9447-9452, 2009; Vecsey, C. G., et al. Journal of Neuroscience, 27, 6128-6140, 2007; Wood, M. A., et al., Learning and Memory, 13, 609-617, 2006; and Wood, M. A., et al. Learning and Memory, 12, 111-119, 2005, the disclosures of each of which are incorporated herein by reference.) Thus, a learning event that produces long-term memory enhances histone acetylation, by increasing HAT and decreasing HDAC activity, to induce specific patterns of gene expression. (See, e.g., Federman, et al., Learn Memory, 16, 600-606, 2009, the disclosure of which is incorporated herein by reference.)

In contrast, HDACs have been shown to be powerful negative regulators of long-term memory processes. Nonspecific HDAC inhibitors have been shown to enhance synaptic plasticity as well as long-term memory. (See, e.g., Levenson et al., J Biol Chem 279:40545-40559, 2004; Bredy & Barad, Learn Mem 15:39-45, 2008; Lattal et al., Behav Neurosci 121:1125-1131, 2007; Vescey et al., J Neurosci 27:6128-6140, 2007; Guan et al., Nature 459:55-60, 2009; Malvaez et al., Biol Psychiatry 67:36-43, 2010; Roozendaal et al., J Neurosci 30:5037-5046, 2010, the disclosures of each of which are incorporated herein by reference.) For example, the HDAC inhibitor sodium butyrate can transform a learning event that does not lead to long-term memory into a learning event that does result in significant long-term memory. (Stefanko et al., Proc Natl Acad Sci USA 106:9447-9452, 2009, the disclosure of which was incorporated herein by reference.) Furthermore, sodium butyrate can also generate a form of long-term memory that persists beyond the point at which normal memory fails. HDAC inhibitors have also been shown to ameliorate cognitive deficits in genetic models of Alzheimer's disease (Fischer et al., Nature 447:178-182, 2007; and Kilgore et al., Neuropsychopharmacology 35:870-880, 2010, the disclosures of each of which are incorporated herein by reference.) These demonstrations of modulating memory via HDAC inhibition provide an indication that there could be therapeutic potential for many cognitive disorders using these techniques. However, because HDACs also impact many other processes in the body, these nonspecific HDAC inhibitors may cause side-effects unrelated to the regulation of memory loss.

What is more, currently the role of individual HDACs in long-term memory formation remains largely unexplored. One study revealed that nonspecific HDAC inhibitors, such as sodium butyrate, inhibit class I HDACs (HDAC1, 2, 3, 8) with little effect on the class IIa HDAC family members (HDAC4, 5, 7, 9). This suggests that inhibition of class I HDACs are critical for the enhancement of cognition observed in many studies. Indeed, forebrain over-expression of HDAC2, but not HDAC1, negatively regulates memory formation. (See, e.g., Guan et al., Nature 459:55-60, 2009, the disclosure of which is incorporated herein by reference.) For example, to date, no studies have examined the function of HDAC3 in memory formation. And yet, HDAC3 is the most highly expressed class I HDAC throughout the brain, including the hippocampus. (See, e.g., Broide et al., J Mol Neurosci 31:47-58, 2007, the disclosure of which is incorporated herein by reference.) HDAC3 alters gene expression within a large complex that contains co-repressors, NCoR and SMRT, as well as class IIa HDACs, like HDAC4. (Guenther et al., Genes Dev 14:1048-1057, 2000; Li et al., Embo J 19:4342-4350, 2000; and Karagianni & Wong, Oncogene 26:5439-5449, 2007, the disclosures of each of which are incorporated herein by reference.) NCoR associates with HDAC3 through the deacetylase activation domain (DAD) of NCoR, and a single amino acid substitution (Y478A) in the NCoR DAD results in a mutant protein that is unable to associate with or activate HDAC3. (Alenghat et al., Nature 456:997-1000, 2008, the disclosures of which are incorporated herein by reference.) In addition, class IIa HDACs may require interaction with HDAC3 for their HDAC activity. (Fischle et al., Mol Cell 9:45-572002).

Accordingly, a need exists to develop a tailored approach that is able to more specifically target and regulate the processes that are involved in long-term memory formation.

### SUMMARY OF THE INVENTION

The current invention provides novel therapeutic methods and systems for the regulation of long-term memory formation which is exemplified by and can be achieved through a variety of genetic and pharmacologic approaches.

In one embodiment, the invention is directed to a method of regulating the transcription required for long-term memory formation for treating a memory disorder by administering a therapeutic amount of a pharmaceutical that down-regulates the functional activity of at least one of HDAC3 and HDAC4 to a patient diagnosed with the memory disorder.

In another embodiment, the invention is directed to a pharmaceutical compound for the treatment of a memory disorder that includes a therapeutically effective amount of at least one medicament that selectively down-regulates the functional activity of both HDAC3 and HDAC4.

In still another embodiment, the invention is directed to a pharmaceutical compound for the treatment of a memory disorder that includes a therapeutically effective amount of at least one medicament that selectively down-regulates the functional activity of HDAC3.

In yet another embodiment, the invention is directed to a pharmaceutical compound for the treatment of a memory disorder that includes a therapeutically effective amount of at least one medicament that selectively inhibits the enzymatic activity of HDAC3.

In still yet another embodiment, the invention is directed to a method of treating a memory disorder that comprises down-regulating the functional activity of HDAC4.

In still yet another embodiment, the invention includes the use of a therapeutically effective amount of a substituted or unsubstituted N-(o-aminophenyl) carboxamide compound.

In still yet another embodiment, the invention includes the use of RGFP136, 109 and/or 966.

In still yet another embodiment, the invention is directed to a method of treating a memory disorder that comprises inserting a point mutation via gene therapy techniques to directly and specifically disrupt the NCoR/HDAC3/HDAC4 complex such that the functional activity of one or both of HDAC3 and/or HDAC4 are down-regulated.

In still yet another embodiment, the invention includes the treatment of a memory disorder including cognitive disorders, neurodegenerative diseases, and aging.

In still yet another embodiment, the invention includes the treatment by extinction of a negative memory, such as addiction or post traumatic stress.

### BRIEF DESCRIPTION OF THE DRAWINGS

The description and claims of the current invention will be more fully understood with reference to the following figures and data graphs, which are presented as exemplary embodiments of the invention and should not be construed as a complete recitation of the scope of the invention, wherein:

FIG. 1, provides a schematic of the interplay between HDAC and memory regulation.

FIG. 2 provides a schematic of illustration of the transcriptional regulation by interactions with HATs and HDACs, where the nucleosome are represented as blue cylinders with DNA tightly wound around them in black, and the dotted lines represent theoretical immediate early gene expression levels after learning with an HDAC inhibitor. Although depicted as separate protein complexes, it should be understood that HATs and HDACs may be found in the same complex.

FIGS. 3A to 3D, provide data results showing that intrahippocampal AAV2/1-Cre infusion in HDAC3^{flox/flox} mice results in a complete, focal deletion of HDAC3 and alters expression of other acetylation markers (images are 4× except the right panels which are 20× magnifications of the regions boxed in white), wherein: (A) representative images showing DAPI labeling and HDAC3 immunoreactivity in hippocampi of AAV2/1-Cre infused HDAC3^{+/+} and HDAC3^{flox/flox} mice, HDAC3 labeling is found throughout CA1, CA3 and the dentate gyrus, and no immunoreactivity is found in the AAV2/1-Cre infusion site of HDAC3^{flox/flox} mice; (B) representative images showing HDAC2 immunoreactivity in hippocampi is unchanged in AAV2/1-Cre infused HDAC3^{flox/flox} mice; (C) HDAC4 immunoreactivity is decreased in the region of the HDAC3 deletion (* indicates p<0.05); and (D) further, acetylation at H4K8 is increased specifically in the AAV2/1-Cre infusion site of HDAC 3^{flox/flox} mice (* indicates p<0.05).

FIGS. 4A to 4C, provide data results showing that c-fos and Nr4a2 expression are increased in the area of focal homozygous deletion of HDAC3 in HDAC3^{flox/flox} mice, wherein: (A) mice received subthreshold training (3 min) in an environment with 2 identical objects; (B) 2 hr following training, brains were collected and sliced to collect 1 mm punches from area of focal deletion of HDAC3 (as confirmed by immunohistochemistry in adjacent slices), quantitative RT-PCR shows that c-fos expression is significantly increased in the dorsal hippocampus of HDAC3^{flox/flox} mice as compared to wildtype littermates (p<0.001); and (C) training induced greater Nr4a2 expression in the dorsal hippocampus of HDAC3^{flox/flox} mice compared with wild-type littermates (n=3 per group; *p<0.02).

FIGS. 5A to 5E, provide data results showing that focal homozygous gene deletion of HDAC3 in the dorsal hippocampus leads to enhanced memory for object location (OLM), which persists at least 7 days, wherein: (A) mice received subthreshold training (3 min) in an environment with 2 identical objects and received a retention test 24 hrs or 7 days later in which one object is moved to a new location; (B) HDAC3^{flox/flox} mice demonstrated significant long-term memory for object location 24 hours after subthreshold training (n=8/group, ** indicates p<0.005); (C) in a different set of mice, the persistence of this enhanced memory was examined, HDAC3^{flox/flox} mice displayed a significant preference for the novel object location compared with HDAC3^{+/+} mice during a 7-day retention test (n=9/group, ** indicates p<0.001); (D) mice received subthreshold training (3 min) in an environment with two identical objects and received a retention test 24 hr later in which one object is replaced with a novel one (ORM); (E) neither HDAC3^{+/+} or HDAC3^{flox/flox}, mice exhibited significant preference for the novel object (n=8 per group.

FIGS. 6A and 6E, provide data results showing that loss of HDAC3/NCoR interaction enhances long-term OLM and ORM but has no effect on short-term memory, wherein: (A) mice received subthreshold training (3 min) in an environment with 2 identical objects and received a retention test (B) 90 min or (C) 24 hr later in which one object is replaced with a novel one; and (B) subthreshold training did not result in significant short-term memory by either genotype when tested 90 min later (n=11-13 per group); (C) DADm mice showed a significant preference for the novel object location 24 hr after training compared with wild types (n=9-10 per group, **p<0.005); (D) mice received subthreshold training (3 min) in an environment with two identical objects and received a retention test 24 h later in which one object is replaced with a novel one (ORM); (E) DADm mice showed a significant preference for the novel object itself 24 hr after training compared with wild types (n=12-18 per group, *p<0.05).

FIGS. 7A to 7D, provide data results showing that intrahippocampal RGFP136 infusions cause alterations in deacetylation enzymes and histone acetylation markers. Images on left are 4×, and 20× magnifications of the regions boxed in white are on the right, wherein: (A) HDAC3 immunoreactivity is unaltered in area of infusion 2 hours after RGFP136 treatment, but not vehicle; (B) representative images show HDAC2 immunoreactivity in dorsal hippocampus is unchanged by drug treatment; (C) HDAC4 nuclear immunoreactivity is decreased in the region of the RGFP136 infusion (* indicates p<0.05); and (D) Acetylation at H4K8 is increased in RGFP136 infused mice compared to those treated with vehicle (* indicates p<0.05).

FIGS. 8A to 8I, provide data results showing that the HDAC inhibitor RGFP136 enhances long-term memory for ORM and OLM following systemic delivery, wherein: (A) mice received subthreshold training (3 min) in an environment with 2 identical objects immediately followed by subcutaneous injection of RGFP136 and received a retention test (B) 24 h or (C) 7 days later in which one object is replaced with a novel one; (B) mice treated with the 30 mg/kg dose exhibited significantly better long-term memory for the familiar object than vehicle-treated controls, while 150 mg/kg treatment resulted in memory no different from vehicle (n=7-9/group, Two-way ANOVA, ** indicates p<0.01); (C) in a different set of mice, the persistence of this enhanced memory was examined, mice receiving subcutaneous injection of RGFP136 (30 mg/kg) exhibited significantly increased exploration of the novel object compared with vehicle-treated mice during a 7-day retention test (n=9-10/group, ** indicates p<0.01); (D) mice received subthreshold training (3 min) in an environment with 2 identical objects immediately followed by a subcutaneous injection of RGFP136 (30 mg/kg) or vehicle and received a retention test (E) 90 min or (F) 24 h later in which one object is moved to a new location; (E) subthreshold training did not result in significant short-term memory after RGFP136 (30 mg/kg); (F) mice treated with the 30 mg/kg RGFP136 exhibited significantly better long-term memory for the familiar object than vehicle-treated controls (n=9-10/group, ** indicates p<0.001); (G) mice received subthreshold training (3 min) in an environment with two identical objects followed immediately by intrahippocampal infusion of RGFP136 and received a retention test 24 h (H) or 7 d (I) later in which one object is moved to a new location; (H) intrahippocampal RGFP136 treatment led to significant preference for the novel object location 24 h after subthreshold training (n_{—}7 per group **p_{—}0.01); and (I) mice receiving intrahippocampal RGFP136 also displayed a significant preference for the novel object location compared with vehicle-treated mice during a 7 d retention test (n_{—}8 per group; **p_{—}0.001).

FIGS. 9A and 9B, provide data results showing that the HDAC inhibitor RGFP136 requires CBP to enhance long-term memory of OLM, wherein: (A) mice received subthreshold training (3 min) in an environment with 2 identical objects immediately followed by intra-hippocampal infusions of RGFP136 (1.25 ng/side) or vehicle (0.5 μL/side) and received a retention test 24 h later in which one object is moved to a new location; and (B) wildtype CBP^{+/+} mice that received intra-hippocampal RGFP136 immediately following training showed significant long-term memory for the object location compared to vehicle-treated mice, while CBP^{KIX/KIX} mice showed no effect of drug treatment (n=5-9/group, Two way ANOVA, ** indicates p<0.001).

FIG. 10, provides data results showing the dose response of RGFP compounds on novel object recognition, where RGFP 109, 136, and 966 doses showed significantly greater preference for the novel object as compared to vehicle (* p<0.05; ** p<0.001), and the 30 mg/kg dose of 109 and 136 had greater object discrimination than lower doses (30 mg/kg vs. 3 mg/kg, †† p<0.01; 30 mg/kg vs. 10 mg/kg, ⁰⁰ p<0.01), whereas RGFP 999, an inactive compound, did not demonstrate a significant preference as compared to vehicle-treated mice.

FIGS. 11A to 11C, provide data showing that Nr4a2 siRNA attenuates the long-term memory enhancement observed in HDAC3^{flox/flox} mice, wherein (A), shows results at 48 h after infusions of Nr4a2 or RISC-free siRNA, HDAC3^{flox/flox} and HDAC3^{+/+} mice received subthreshold training (3 min) in an environment with two identical objects and received a retention test 24 h later in which one object is moved to a new location; (B), HDAC3^{flox/flox} mice infused with RISC free (n=10) exhibited significant memory for object location compared with HDAC3^{+/+} mice (††p=0.001), which was blocked by Nr4a2 siRNA treatment (n=9-10 per group; **p<0.001); and (C), at 2 h after testing, quantitative RT-PCR shows that Nr4a2 siRNA treatment significantly reduced Nr4a2 expression in both HDAC3^{flox/flox} and HDAC3^{+/+} mice (n=3 per group; **p<0.001 and *p<0.05 vs respective RISC-free siRNA controls), and HDAC3^{flox/flox} mice also exhibited an increased induction of Nr4a2 mRNA after the long-term memory test (††p=0.002 vs HDAC3^{+/+} RISC free).

FIG. 12, provides data showing RGFP 966 dose dependently facilitates extinction of cocaine conditioned place preference (CPP), where CPP score indicates preference by mean±S.E.M. of time in CS+ minus CS− compartment, and all mice displayed a significant preference for the cocaine-paired compartment following conditioning (Posttest). Treatment with RGFP 966 (10 mg/kg, s.c.) immediately following Posttest resulted in rapid extinction of this preference as seen on the following extinction days (Ext2 and Ext3). *p<0.05 vs. Veh, †p<0.05 vs. 3 mg/kg 966, n=12/group.

FIG. 13, provides data showing RGFP 966 dose dependently blocks cocaine-primed reinstatement of drug-seeking behavior, where CPP score indicates preference by mean±S.E.M. of time in CS+ minus CS− compartment, and all groups extinguished the preference for the CS+ compartment by extinction day 6, and wherein the following day, animals received a cocaine prime (10 mg/kg, i.p.) and were placed in the test chamber (Reinstatement). Mice that had previously received vehicle and 3 mg/kg 966 showed a significant preference for the cocaine-paired compartment (paired t-test, * p<0.03 vs. Ext6), demonstrating that cocaine was able to reinstate cocaine-seeking behavior. However, mice that had previously received 10 mg/kg 966 during extinction did not reinstate a preference, thus extinction was refractive to reinstatement (†† p<0.01 vs. vehicle, n=8/group).

FIG. 14, provides data results showing that RGFP 966 treatment immediately following conditioning sessions does not alter cocaine conditioned place preference, wherein mice were conditioned with two pairings of a low dose of cocaine (5 mg/kg, i.p.) and saline, and following each conditioning session, mice received RGFP966 (10 mg/kg, s.c.) or vehicle. All animals displayed a significant conditioned place preference (Posttest), and RGFP treatment did not alter this behavior (* p<0.02**p<0.001 vs. pretest, n=14/group).

### DETAILED DESCRIPTION OF THE INVENTION

The current invention is generally directed to a methodology and therapy for the treatment and regulation of memory function. The invention identifies specific HDAC, and in particular, HDAC3 and HDAC4 as negative regulators of memory formation and specifically targets one or both HDAC3 and HDAC4 for down-regulation. It has been determined that by specifically targeting HDAC3 and HDAC4 with either gene therapies or small molecule inhibitors it is possible to provide a powerful therapeutic approach to facilitate gene expression during memory formation that can lead to the regulation and treatment of memory disorders.

Background on Memory and HDACs

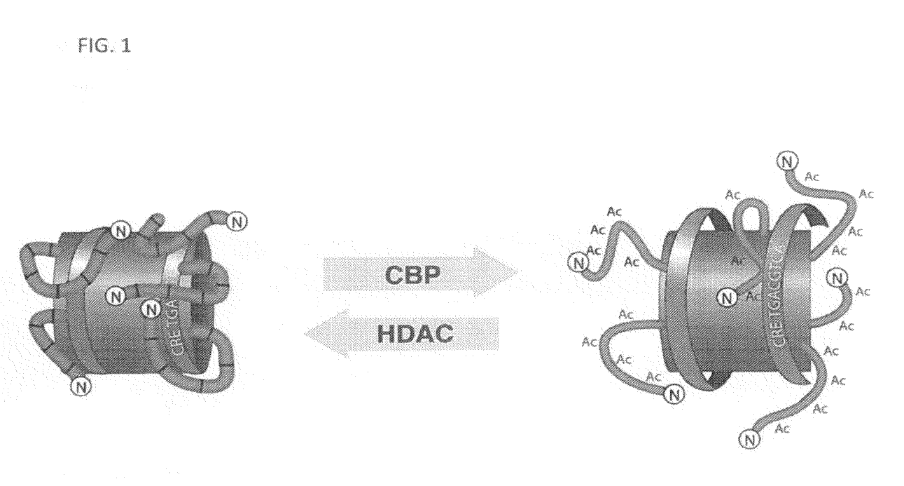

Before describing the invention in detail, it is necessary to understand the therapeutic target. For over four decades scientists have known that long-term memory formation requires gene expression. Gene expression is dynamically regulated by chromatin modifications on histone tails, such as acetylation. In general, histone acetylation promotes transcription, whereas histone deacetylation negatively regulates transcription. As shown diagrammatically in FIG. 1, the interplay between histone acetyltransferases (HATs) and histone deacetylases (HDACs) is pivotal for the regulation of gene expression required for long-term memory processes. However, previously very little was known about the role of individual HDACs in learning and memory. One major problem is identifying which HDACs are involved in negatively regulating memory formation. Previous published studies have shown that non-specific inhibitors modulate memory.

HDACs are grouped into four classes based on sequence homology with yeast factors and domain organization. All classes are dependent on zinc for their catalytic activity except for the sirtuins (Class III) which are structurally unrelated NAD-dependent enzymes and will not be discussed in this review. Class I, comprised of HDACs 1, 2, 3, and 8, share homology with yeast RPD3 protein. This group contains nuclear localization signal (NLS) and lack a nuclear export signal (NES), with the exception of HDAC3 which can be found in the nucleus and cytoplasm (Gregoretti, I. V., et al., Journal of Molecular Biology, 338, 17-31, 2004, the disclosure of which is incorporated herein by reference). Class II HDACs resemble yeast protein HDA1 and are separated by domain organization into IIa (HDACs 4, 5, 7, and 9) and IIb (HDACs 6 and 10). This class contains NLS and NES for phosphorylation-regulated shuttling between the cytoplasm and nucleus as well as additional regulatory domains. HDAC3 has been shown to interact with most of the Class II proteins (HDAC4, 5, 7, and 10). (See, Fischle, W., et al. Molecular Cell, 9, 45-57, 2002; and Tong, J. J., et al., Nucleic Acids Research, 30, 1114-1123, 2002, the disclosures of each of which are incorporated herein by reference.) HDAC11 is the sole member of Class IV, and has been found primarily in the nucleus in complexes with HDAC6 (Gao, L., et al., Journal of Biological Chemistry, 277, 25748-25755, 2002, the disclosure of which is incorporated herein by reference). HDAC11 has similarities with both Class I and II HDACs, but likely has a unique physiological role.

Currently, the role of individual HDACs in long-term memory formation remains largely unexplored except for few recent studies. HDAC5 was the first discrete HDAC to be implicated as a negative regulator of long-term synaptic plasticity. Recruitment of HDAC5 to the C/EBP promoter repressed transcription and blocked long-term facilitation in aplysia (Guan, Z., et al., Cell, 111 483-493, 2002, the disclosure of which is incorporated herein by reference). Further, mice lacking HDAC5 show enhanced reward learning in cocaine conditioned place preference (Renthal, W., et al. Neuron, 56, 517-529, 2007, the disclosure of which is incorporated herein by reference). Conversely, over-expression of HDAC4 or HDAC5 attenuated the expression of cocaine conditioned place preference, further supporting their role as negative regulators of reward-associated memory (Kumar, A., et al. Neuron, 48, 303-314., 2005, the disclosure of which is incorporated herein by reference). However, it was recently shown that purified HDAC4 and HDAC5 have little to no catalytic activity on canonical HDAC substrates containing acetyl-lysines (Lahm, A., Proceedings of the National Academy of Sciences USA, 104, 17335-17340., 2007, the disclosure of which is incorporated herein by reference). There is mounting evidence that Class IIa HDACs function in vivo by interacting with Class I HDACs, which have very potent HDAC activity, and other co-repressors to form multi-protein complexes (See, Fischle et al., 2002 and Lahm et al., 2007, cited above). These findings strongly suggest that future studies should include examination of not only an individual HDAC, but consider the co-repressor complex as a whole to determine how gene expression required for long-term memory formation is being modulated.

Most of the evidence for the contribution of HDACs in learning and memory comes from pharmacological studies. HDAC inhibitors sodium butyrate (NaBut), valproate and suberoylanilide hydroxamic acid (SAHA) were thought to non-specifically block Class I, IIa and Ilb, but not Class III, HDACs. However, a recent biochemical analysis of the in vitro activities of recombinantly expressed, purified HDACs 1-9 demonstrated that those drugs are potent inhibitors of Class I, but not Class IIa and IIb, HDACs (Kilgore, M., et al. Neuropsychopharmacology, 35, 870-880, 2010, the disclosure of which is incorporated herein by reference). These findings suggest that it is the Class I HDACs that are critical for regulating long-term memory processes. Indeed, recent work on individual Class I HDACs supports this hypothesis. Forebrain over-expression of HDAC2, but not HDAC1, caused impaired memory formation and synapse formation (Guan, J. S., et al., Nature, 459, 55-60, 2009, the disclosure of which is incorporated herein by reference). Conversely, loss of HDAC2 resulted in enhanced memory formation and synaptic plasticity. Further, HDAC2, but not HDAC1, was shown to be associated with the promoters of several genes implicated in plasticity and learning, and so it is likely that removal of this negative regulator would allow for greater learning-induced gene expression.

However, no study to date has demonstrated which specific HDACs play a role in memory regulation. As a result, there has been no ability to create a tailored methodology that can effectively target the regulation of memory function without simultaneously impacting many other processes that are regulated by HDACs.

### Expression and Function of HDAC3

HDAC3 is expressed in many tissues throughout the body, including the brain. (Mahlknecht, U., et al., Biochemical and Biophysical Research Communications, 263, 482-490, 1999, the disclosure of which is incorporated herein by reference). It is the most highly expressed Class I HDAC in the brain with greatest expression in the hippocampus, cortex, and cerebellum. (Broide et al., J Mol Neurosci 31:47-58, 2007 the disclosure of which is incorporated herein by reference.) For example, while HDAC3 is predominantly expressed in neurons, it is also one of the few HDACs localized in oligodendrocytes (Broide et al., 2007 cited above; and Shen, S., et al., Journal of Cell Biology, 169, 577-589, 2005, the disclosures of which are incorporated herein by reference), and while its primary localization is in the nucleus, HDAC3 can also be found in the cytoplasm and at the plasma membrane (Longworth, M. S., & Laimins, L. A., Oncogene, 25, 4495-4500, 2006; and Takami, Y., & Nakayama, T. Journal of Biological Chemistry, 275, 16191-16201, 2000, the disclosures of which are incorporated herein by reference). However, no study to date has examined the role of HDAC3 in the brain.

HDAC3 catalytic activity can be regulated by phosphorylation at the serine 424 residue of the C-terminal domain (Zhang, X., et al., Genes & Development, 19, 827-839, 2005, the disclosure of which is incorporated herein by reference). Casein kinase 2 phosphorylation of HDAC3 at this site has been shown to increase the basal enzymatic activity, whereas protein phosphatase 4 has the inverse effect (Zhang et al., 2005, cited above). Although phosphorylation can alter activity of HDAC3, it has not been found to alter subcellular localization or protein interactions (Jeyakumar, et al., Journal of Biological Chemistry, 282, 9312-9322, 2007; and Zhang et al., 2005, cited above, the disclosures of which are incorporated herein by reference). Also, an oligomerization domain has been identified in the N-terminal by which the protein can self-associate to form dimers and trimers (Yang, W. M., et al., Journal of Biological Chemistry, 277, 9447-9454, 2002, the disclosure of which is incorporated herein by reference). However, recombinant HDAC3 alone has no HDAC function (Guenther, et al., Molecular and Cellular Biology, 21, 6091-6101, 2001, the disclosure of which is incorporated herein by reference). HDAC3 must be properly folded by TCP-1 ring complex and then bound to co-repressors NCoR (nuclear receptor co-repressor) or SMRT (silencing mediator of retinoic acid and thyroid hormone receptor) to form an active enzyme complex (Guenther, M. G., et al., Genes & Development, 16, 3130-3135, 2002, the disclosure of which is incorporated herein by reference).

HDAC3 forms a stable multi-protein complex with co-repressors NCoR and SMRT in order to regulate transcription of genes as well as other nontranscriptional functions. Three different binding sites on NCoR/SMRT are associated with HDAC3 (Wen, Y. D., et al., Proceedings of the National Academy of Sciences USA, 97, 7202-7207, 2000, the disclosure of which is incorporated herein by reference). One site important for HDAC3 activity is the deacetylase domain (DAD) of NCoR/SMRT, which binds both the amino and carboxy termini of HDAC3 and transforms HDAC3 into a four-helical structure (Codina, A., et al., Proceedings of the National Academy of Sciences USA, 102, 6009-6014, 2005; and Guenther et al., 2001, cited above, the disclosure of which is incorporated herein by reference). HDAC3 is the primary HDAC enzyme in NCoR/SMRT complexes, however other HDACs or HDAC complexes can be recruited in a transcription factor-specific or context-specific manner by less stable interactions with NCoR/SMRT (Fischle et al., 2001, cited above; and Huang, E. Y., et al., Genes & Development, 14, 45-54, 2000, the disclosure of which is incorporated herein by reference).

This has been best described of the Class II HDACs. Class IIa HDACs (HDAC4 and 5) are found to directly interact with the RD3 domain of NCoR/SMRT, a distinct domain from HDAC3, and become part of the repressor complex (Fischle et al., 2001; Huang et al., 2000; and Wen et al., 2000, cited above). In addition, Class II HDACs (4, 5, 7, and 10) have been shown to interact with HDAC3, but not HDAC1 or 2 (Fischle et al., 2001, 2002; and Huang et al., 2000, cited above). Specifically, HDAC4 coimmunoprecipitates with HDAC3 via its C-terminal domain and disruption of this interaction results in loss of observed HDAC activity. Further, it has been suggested that the enzymatic activities of Class IIa HDACs rely on interactions with HDAC3 and NCoR/SMRT (Fischle et al., 2001; and Huang et al., 2000, cited above). Purified HDAC4 and 5 have little tonocatalytic activity on canonical HDAC substrates containing acetyl-lysines (Lahm et al., 2007, cited above). However, HDAC4 or 5 associated with HDAC3 and/or NCoR results in observable deacetylase activity which is disrupted by mutations in these interaction domains. Thus, Class IIa HDACs likely function in vivo by interacting with HDAC3, which has potent HDAC activity, as part of a co-repressor multi-protein complex (Fischle et al., 2002; and Lahm et al., 2007, cited above).

Previous in vitro studies have shown that HDAC3 and HDAC4 interact with each other in large complexes (Grozinger C M, and Schreiber S L, Proc Natl Acad Sci USA 97:7835-7840, 2000; and Fischle et al., 2002, cited above, the disclosures of which are incorporated herein by reference). In other words, interactions between HDAC3 and HDAC4 create a functional complex involved in transcriptional regulation. HDAC4 and HDAC5 are considered to be in the “inactive state” until they are bound to HDAC3, an interaction necessary for their catalytic activity. (See, Fischle et al., 2002, cited above.) A study by Lahm et al. supported previous findings that class IIa HDACs (HDAC4, 5, 7, and 9) are inactive on acetylated substrates, thus distinguishing them from class I HDACs (HDAC1, 2, 3, and 8). (Lahm et al., Proc Natl Acad Sci USA 104:17335-17340, 2007, the disclosure of which is incorporated herein by reference.) This has called into question the catalytic activity of class IIa HDACs; or an equally reasonable idea is that the natural substrate of these enzymes has not been identified. In any case, the interaction between HDAC4 and HDAC3 is facilitated by co-repressor proteins NCoR and SMRT, which form a large complex with HDACs and other proteins. (Fischle et al., 2002, cited above.) HDAC4 and HDAC3 bind independently to different domains of SMRT and NCoR, but the proximity allows for interactions of these proteins.

Lahm et al. showed that a critical residue for HDCA3 activity is a tyrosine at amino acid 298, which if mutated to a histidine (Y298H) completely abrogates enzymatic function. (Lahm et al., 2007, cited above.) Although not to be bound by theory, HDAC4 and other class IIa enzymes normally have a histidine at this position, which provides a potential reason why HDAC4 has such poor enzymatic activity on traditional substrates. Commonly used HDAC inhibitors, such as VPA, sodium butyrate, phenylbutyrate, and SAHA, have been shown to greatly inhibit class I HDACs (HDAC1, 2, 3, 8) with little effect on the class IIa HDAC family members (HDAC4, 5, 7, 9). (See, Kilgore et al., Neuropsychopharmacology 35:870-880, 2010, the disclosure of which is incorporated herein by reference.) This suggests that inhibition of class I HDACs are critical for the reported effects of HDAC inhibition, such as the enhancement of cognition.

Indeed, HDAC2 has been implicated as a specific target to negatively regulate memory formation (Guan et al., 2009, cited above). Over-expression of HDAC2, but not HDAC1, in the forebrain caused reductions in synaptic plasticity and corresponding learning impairments, while the converse was found in HDAC2-deficient mice. However, despite all these studies, thus far the mechanism of memory regulation has not been identified, and, as such, no therapeutic or therapeutic method has been proposed that would allow for the regulation of memory function in a patient suffering from memory dysfunction.

### SUMMARY OF THE INVENTIVE METHODOLOGY

In developing the therapies of the current invention, a number of gene and pharmaceutical techniques were used to explore the role of individual HDACs and in particular HDAC3 in learning and memory. It has been surprisingly discovered that HDAC3 is a negative regulator of memory formation, and that selective down-regulation of HDAC3 and HDAC4 provide a therapeutic means of treating memory disorders by regulating the gene transcription required for long-term memory. In particular, as will be discussed in greater detail in the exemplary embodiments provided below:
HDAC3-FLOX genetically modified mice in combination with AAV expressing Cre recombinase were used to generate focal homozygous deletions of HDAC3 in area CA1 of the dorsal hippocampus; and
Several selective inhibitors of HDAC3, including RGFP136, RGFP109, and RGFP966 produced by the Repligen Corporation was delivered either systemically or site-specifically to the dorsal hippocampus immediately after training.
   Both the focal deletion of HDAC3, as well as the down-regulation of HDAC3 and HDAC4 via the selective inhibitors, are shown to significantly enhanced long-term memory in a persistent manner.

In addition, immunohistochemistry studies show that focal deletion or intrahippocampal delivery of the HDAC3 inhibitor resulted in increased histone acetylation. In addition, subthreshold training activated c-fos gene expression greater in neurons lacking HDAC3. To further explore the role of HDAC3/4 as a negative regulator of long-term memory formation, genetically modified mice carrying a point mutation in NCoR, which disrupts NCoR-HDAC3 interactions resulting in loss of HDAC3 activity, were examined. Homozygous NCoR knockin mice also exhibited significantly enhanced long-term memory. Finally, expression of nuclear receptor subfamily 4 group A, member 2 (Nr4a2) and c-fos was significantly increased in the hippocampus of HDAC3-FLOX mice compared with wild-type controls. Moreover, memory enhancements observed in HDAC3-FLOX mice were abolished by intrahippocampal delivery of Nr4a2 small interfering RNA, suggesting a mechanism by which HDAC3 negatively regulates memory formation, and which can be targeted by specific inhibitors and gene therapies. Together, these findings demonstrate a critical role for HDAC3 in the molecular mechanisms underlying long-term memory formation.

Mechanism of Inventive Therapy

As it has been discussed, HDAC3 can repress CBP function by deacetylation (Chuang, H. C., et al., Nucleic Acids Research, 34, 1459-1469, 2006; and Gregoire, S., et al. Molecular and Cellular Biology, 27, 1280-1295, 2007, the disclosures of which are incorporated herein by reference). As such, and not to be bound by theory, but is likely that HDAC3 inhibition allows greater CREB-CBP interactions to enhance gene transcription necessary for memory formation. As will be described in greater detail below, this hypothesis was tested using genetically modified CBP mutant mice carrying a triple point mutation in the phospho-CREB (KIX) binding domain of CBP (CBP^{KIX/KIX} mice; Kaspar, B. K., et al. Proceedings of the National Academy of Sciences USA, 99, 2320-2325, 2002, the disclosure of which is incorporated herein by reference). Also it is demonstrated that HDAC inhibition enhances hippocampal synaptic plasticity in wildtype but not CBP^{KIX/KIX} mice, suggesting enhancement via HDAC inhibition requires hippocampal CREB:CBP interaction (Vecsey, C. G., et al. Journal of Neuroscience, 27, 6128-6140, 2007, the disclosure of which is incorporated herein by reference). Also, CBP^{KIX/KIX} mice have deficits in long-term memory formation of a hippocampus-dependent task (Haettig, J., et al., Learning and Memory, 18, 71-79, 2011, the disclosure of which is incorporated herein by reference). Finally, intrahippocampal delivery of selective inhibitors, such as, for example, RGFP136 resulted in long-term memory after subthreshold training in CBP^{+/+} mice, but not CBP^{KIX/KIX} littermates (McQuown, S. C., et al. Journal of Neuroscience, 31, 764-774, 2011, the disclosure of which is incorporated herein by reference). These results indicate that RGFP136, like sodium butyrate and trichostatin A, enhances long-term memory through a CBP-dependent mechanism. This appears to be a fundamental mechanism by which HDAC inhibitors modulate hippocampal synaptic plasticity and hippocampus-dependent long-term memory, and strongly suggest that HDAC inhibitors (even only Class I specific inhibitors) modulate memory via a specific mechanism.

These results suggest that HDACs and associated co-repressors form complexes (or molecular brake pads) that normally maintain specific genes in a silent state and sufficiently strong activity-dependent signaling is required to temporarily remove these complexes (or brake pads) to activate gene expression required for long-term memory formation. Thus, these repressor complexes (or brake pads) are always on, except during important signaling events triggering specific gene expression profiles for cellular function. If this hypothesis is correct several features would be predicted, which are discussed below.

Genomic DNA in its relaxed form would extend approximately two meters, which needs to fit into a 6 lm diameter nucleus. To achieve this incredible level of compaction, genomic DNA goes through multiple levels of organization resulting in approximately a 10,000 fold compaction. “10,000 fold” is an extremely difficult idea to grasp, but it becomes readily clear that accessing and indexing genes required for long-term memory processes is a remarkable achievement. The point is that the molecular machinery involved in this organization and compaction of genomic DNA is part and parcel to accessing and indexing genes. It helps to consider this before exploring how genes are turned on for long-term memory formation—it's not just as simple as loading RNA pol II.

One simple prediction is that HDACs and associated co-repressors forming “molecular brake pads” are normally engaged in silencing gene expression because they are normally involved in the compaction of chromatin structure. However, there are many mechanisms of genomic DNA compaction (polycomb, etc.), so what makes HDACs and associated co-repressors unique? First, HDACs and associated co-repressors are preferentially found at actively transcribed genes in a constant interplay with HATs and RNA pol II to regulate gene expression. A recent genome-wide mapping of HATs and HDACs found that both are found at active genes with acetylated histones and both are targeted to transcribed regions of active genes by phosphorylated RNA pol II (Wang, Z., et al., Cell, 138, 1019-1031, 2009, the disclosure of which is incorporated herein by reference). The authors extend the interpretation of their findings to conclude that the majority of HDACs function to reset chromatin by removing acetylation at active genes. These results support the idea of HDACs and associated co-repressors functioning as “molecular brake pads” at actively transcribed genes as they are primarily found at actively transcribed genes and reset their state of expression.

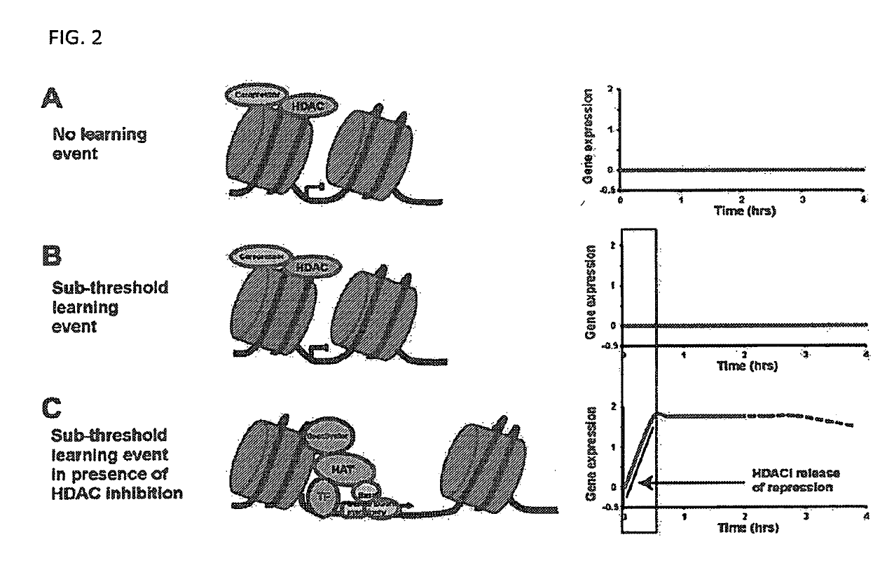

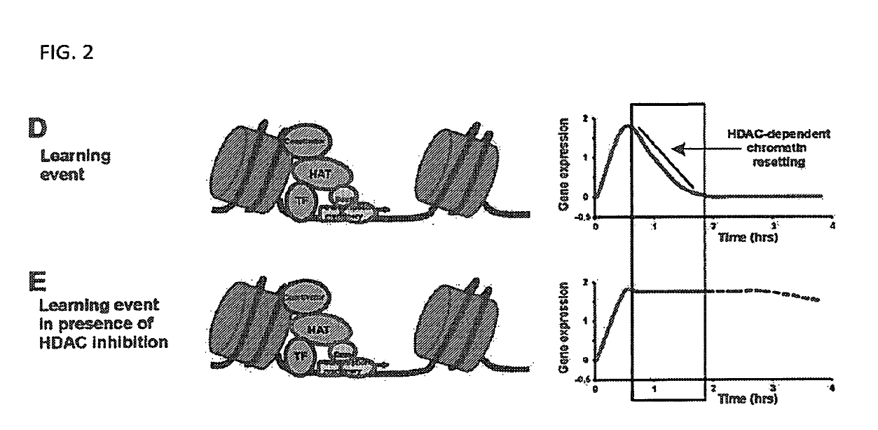

Another simple prediction is that inhibition of these molecular brake pad complexes should have specific consequences on activity-dependent transcription and long-term memory (see, FIG. 2). If these molecular brake pad complexes serve to reset chromatin and silence gene expression following activity-dependent signaling, then prohibiting the molecular brake pads from re-engaging may be predicted to prolong gene expression beyond the point it would normally following a learning event. This has been observed in a study by Vecsey et al. (2007), cited above, in which mice were fit with intrahippocampal cannulae, subject to contextual fear conditioning, and then immediately after training injected with an HDAC inhibitor. Two hours after training, hippocampi were collected and gene expression was examined. At a point when immediate early genes are normally turned off, the immediate early gene and transcription factor Nr4a2 had maintained expression, which was associated with increased histone acetylation at its promoter (Vecsey et al., 2007, cited above). HDAC inhibition alone had no effect on the genes examined and contextual fear conditioning alone did not result in maintained gene expression at 2 h post-training. Furthermore, out of about a dozen genes examined, only Nr4a2 and Nr4a1 had maintained gene expression. These results demonstrate that HDAC inhibition may prevent the resetting of chromatin structure by molecular brake pad complexes, resulting in maintained gene expression beyond the point normally observed after learning.

Does maintained gene expression result in enhanced long-term memory? Does maintained gene expression transform a learning event that does not normally result in short- or long-term memory into an event that does? The studies presented herein have demonstrated remarkable effects on the modulation of memory by HDAC inhibition. In particular, one simple prediction of the molecular brake pad hypothesis is that if the brake pads are removed, then a subthreshold stimulus should result in long-term potentiation (LTP) and long-term memory. With regard to synaptic plasticity, Vecsey et al. (2007), cited above, showed that a stimulus that normally induces a transient, transcription independent form of LTP, can be transformed into a stable, transcription-dependent form of LTP in the presence of HDAC inhibition. With regard to long-term memory, Stefanko et al. (2009), cited above, showed that a subthreshold learning period of three minutes, which does not result in observable short- or long-term memory, does result in robust long-term memory in the presence of HDAC inhibition. Similarly, HDAC3-FLOX mice with a focal homozygous deletion of HDAC3 in the dorsal hippocampus also exhibit robust long-term memory for object location following a subthreshold training period (McQuown et al., 2011, cited above).

Indeed, Haettig et al. (2011) and McQuown et al. (2011), cited above, recently showed that HDAC inhibition modulates hippocampus-dependent long-term memory in a CBP-dependent manner. This supports the interplay between HDACs and HATs as suggested by Wang et al. (2009), cited above, in regulating actively transcribed genes. More importantly, there is strong evidence presented in this disclosure, and described in detail below, demonstrating that removal of molecular brake pad complexes results in remarkable effects on long-term memory predicted by this hypothesis.

Accordingly, the current invention identifies the role of HDAC3 in long-term memory as a negative regulator of memory formation using a combined genetic and pharmacologic approach. In addition, the invention demonstrates that targeting HDAC3 and HDAC4 with either gene therapies or small molecule inhibitors provides a powerful therapeutic approach to facilitate gene expression during memory formation. Such HDAC3/4 down-regulation represents a novel therapy and the gene therapies and small molecule inhibitors that this invention demonstrates can be used as therapeutic techniques to address cognitive impairments associated with normal aging, neurodegenerative diseases, extinction of memories associated with post-traumatic stress disorder or addiction, and the facilitation of memory processes in general.

Accordingly, in one embodiment, the method of the current invention comprises administering a therapeutically effective amount of a pharmaceutical composition containing at least one HDAC suppressor that selectively down-regulates one or both of HDAC3 and HDAC4 to a patient suffering from a memory dysfunction. Details concerning the inhibitor, the pharmaceutical form the inhibitor can take, the method of administration, the types of memory dysfunctions that can be targeted are described in the description and the exemplary embodiments set forth below.

### Details of the Inhibitor

Turning to the inhibitor itself, the invention is directed to a type of small molecule inhibitor that blocks histone deacetylase (HDAC) function. More particularly, the invention is directed to inhibitors that have been specifically designed to be selective for down-regulation of one or both of HDAC3 or HDAC4. The results of the inventive studies demonstrate that such inhibitors, when administered in therapeutically effective amounts, can enhance long-term memory formation as well as the persistence of long-term memory. In other words, the inhibitor can transform a learning event that did not lead to short- or long-term memory into an event that does result in long-term memory. The administration of such a HDAC3/4 selective down-regulation can also generate a form of long-term memory that persists beyond the point at which normal memory fails.

Although any suitable HDAC3/4 selective down-regulator may be used with the current invention, one particularly preferred inhibitor is a new class of HDAC inhibitor based on substituted or unsubstituted N-(o-aminophenyl) carboxamides. (See, e.g., Chou et al., J Biol Chem 283:35402-35409, 2008; Xu et al., Chem Biol 16:980-989, 2009; and Rai et al., PLoS One 5:e8825, 2010, the disclosures of each of which are incorporated herein by reference.) These inhibitors are slow-on/slow-off, competitive tight-binding inhibitors that specifically target class I HDACs, with the greatest inhibitory effect on HDAC3. (See, Chou et al., 2008; and Xu et al., 2009, cited above.)

Some particularly preferred compounds, used in the exemplary embodiments herein, include, for example, RGFP136, 109 and 966 as well as closely related structures produced by Repligen Corporation. (See, e.g., Rai et al., cited above.) Related structures of similar compounds are also published in Xu et al. (2009; cited above.) These compounds differ from other HDAC inhibitors in their unique selectivity for HDAC3.

It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

The therapeutically effective amount of active agent to be included in the pharmaceutical composition of the invention depends, in each case, upon several factors, e.g., the type, size and condition of the patient to be treated, the intended mode of administration, the capacity of the patient to incorporate the intended dosage form, etc. Generally, an amount of active agent is included in each dosage form to provide from about 0.1 to about 250 mg/kg, and preferably from about 0.1 to about 100 mg/kg. Specific examples of these calculations can be found in the exemplary embodiments, set forth below.

It will be understood that the inhibitor when used in accordance with the current invention has tremendous therapeutic potential for ameliorating memory impairments associated with cognitive disorders, neurodegenerative diseases, aging, or likely any condition resulting in impaired learning and memory. In addition, this class of inhibitors can facilitate the extinction of drug seeking behavior. Extinction is a form of learning, which further supports our main finding that this class of inhibitors enhances learning and memory.

### Details of Gene Therapy

The invention also describes novel gene therapeutics to allow for the regulation of gene-expression related to memory formation. As will be described below in the exemplary embodiment, experimental results from genetically modified HDAC3 mutant mice demonstrate that down-regulation of HDAC3 results in enhanced long-term memory processes. Data shows that the loss of HDAC3 leads to the mislocalization and down-regulation of HDAC4 as well. Thus, the effect of down-regulating HDAC3 on long-term memory is likely via disruption of the HDAC3/HDAC4 protein complex. This complex also contains the co-regulator NCoR and our data from genetically modified NCoR mutant mice supports the idea that disrupting this complex enhances long-term memory processes. NCoR mutant mice express a mutant protein carrying a single amino acid substitution (a point mutation), which disrupts its interaction with HDAC3 (Alenghat, T., et al., Nature, 456, 997-1000, 2008, the disclosure of which is incorporated herein by reference).

These findings strongly support the idea that delivering NCoR with this point mutation via gene therapy techniques would directly and specifically disrupt the NCoR/HDAC3/HDAC4 complex resulting in enhanced learning and memory. Such gene therapy strategies would not be limited to NCoR, but could also involve any mechanism by which disruption of this complex can be achieved in order to facilitate learning and memory, especially long-term memory processes.

Although only specific embodiments of the invention are discussed above and in the examples below, it should be understood that the unique memory regulation methodology of the current invention allows for a number of applications including, for example, ameliorating memory impairments associated with cognitive disorders, neurodegenerative diseases, aging, or likely any condition resulting in impaired learning and memory. In addition the methodology and therapeutics of the current invention may be used to for a number of memory extinction treatments. For example, as will be discussed in greater detail below, the invention may be used to treat addiction.

### EXEMPLARY EMBODIMENTS

The present invention will now be illustrated by way of the following examples, which are exemplary in nature and are not to be considered to limit the scope of the invention.

The examples described herein examined the role of HDAC3 in learning and memory using three different approaches (McQuown et al., 2011, cited above). First, HDAC3flox/flox mice were infused with AAV-Cre recombinase into the dorsal hippocampus to create a homozygous focal deletion of HDAC3. Another genetic approach used was the DADm mouse that has a single amino acid substitution in the DAD domain that disrupts HDAC3 binding to NCoR (Alenghat et al., 2008, cited above). And last, a series of pharmacological inhibitors with greatest inhibition of HDAC3, were used (Rai et al., 2010, cited above). All three approaches lead to facilitated long-term memory formation after a subthreshold training period in the novel object recognition task (McQuown et al., 2011, cited above), while this subthreshold training period failed to yield 24-h long-term memory in control animals. As will be described below, these behavioral findings suggest that HDAC3 is a critical negative regulator of long-term memory formation.

### Methods & Materials

Subjects and Surgical Procedures:

HDAC3 floxed C57BL/6 mice were generated with loxP sites flanking exon 4 through exon 7 of the HDAC3 gene, a region required for the catalytic activity of the enzyme. These mice were generated by the lab of Dr. Mitch Lazar at the University of Pennsylvania. Targeted mutagenesis was performed in C57BL/6 ES cells and HDAC3-FLOX mice have been maintained on a C57BL/6 background.

To generate a focal deletion, mice were infused with adeno associated virus expressing Cre-recombinase (AAV2/1-Cre; Penn Vector Core, University of Pennsylvania, Philadelphia, Pa.) 2 weeks prior to behavioral procedures. Mice were anesthetized with isoflurane and placed in a digital Just For Mice stereotax (Stoelting, Wood Dale, Ill.). 1.0 μl of virus was injected at a rate of 6 μl/hr via an infusion needle positioned in the dorsal CA1 area of the hippocampus (antereoposterior (AP) −2.0; mediolateral (ML) ±1.5; dorsoventral (DV) −1.5). NCoR homozygous knock-in mice (referred to as DADm mice) were generated on C57BL/6 background using homologous recombination to incorporate a single amino acid substitution (Y478A) in the NCoR deacetylase activation domain (DAD). DADm mice are fully described in Alenghat et al. (2008), cited above. CBP^{KIX/KIX} homozygous knock-in mice were generated as previously described (Kasper et al., 2002). These mice carry a triple-point mutation in the phospho-CREB (KIX) binding domain of CBP.

For the Nr4a2 knockdown experiment, SMART pool small interfering RNAs (siRNAs) (Dharmacon) targeted against Nr4a2 were prepared with jetSl (Polyplus Transfection) at a final concentration of 4_M before injection. Intrahippocampal infusions of Nr4a2 siRNA or RNA-induced silencing complex (RISC)-free control siRNA were performed similarly to the infusion procedure above. These surgeries were performed on hippocampal AAV-Cre-infused HDAC3^{flox/flox} and HDAC3^{+/+} mice 2 d before training. Immunohistochemistry and quantitative reverse transcription-PCR were used to confirm focal deletions and siRNA knockdown, respectively, and lack of either was used as criteria for exclusion from those experimental groups. For all other experiments, C57BL/6J male mice were acquired from Jackson Laboratory (Bar Harbor, Me.).

Mice were anesthetized with isoflurane and bilateral cannulae (Plastics One) aimed at the dorsal hippocampus were stereotaxically implanted (AP −1.7; ML ±1.2; DV −1.5). For all experiments, mice were 8-12 weeks old and had ad libitum access to food and water in their home cages. Lights were maintained on a 12 hour light/dark cycle, with all behavioral testing carried out during the light portion of the cycle. All experiments were conducted according to National Institutes of Health guidelines for animal care and use and were approved by the Institutional Animal Care and Use Committee of the University of California, Irvine.

Drugs:

The selective inhibitors: RGFP136 (C₂₀H₂₄FN₃O₂; N-(6-(2-amino-4-fluorophenylamino)-6-oxohexyl)-4-methylbenzamide), RGFP109 (C₂₀H₂₅N₃O₂; N-(6-((2-aminophenyl)amino)-6-oxohexyl)-4-methylbenzamide), and RGFP966 (C₂₁H₁₉FN₄O; (E)-N-(2-amino-4-fluorophenyl)-3-(1-cinnamyl-1H-pyrazol-4-yl)acrylamide), were provided by Repligen Corporation and has been previously described in Rai et al. (2010). Drug was dissolved in DMSO and diluted in a vehicle of 20% glycerol, 20% PEG 400, 20% propylene glycol, and 100 mM sodium acetate (pH 5.4). The final DMSO concentration was no greater than 10%, and the same concentration of DMSO was included in vehicle injections. For experiments, doses were 1.25 ng per side (0.5 μL volume) for intrahippocampal infusion and 30 or 150 mg/kg i.p. for systemic administration.

Immunohistochemistry:

Two weeks after mice were infused with AAV-Cre or two hours after hippocampal infusion of the inhibitor, mice were anesthetized deeply with sodium pentobarbital (100 mg/kg, i.p.) and perfused transcardially with ice-cold PBS, pH 7.4, followed by ice-cold 4% paraformaldehyde in PBS, pH 7.4, using a peristaltic perfusion pump (Fisher Scientific). The brains were removed, postfixed overnight at 4° C., and then transferred to 30% sucrose for 48 hr at 4° C. Brains were frozen and cryocut to 20 μm coronal slices, and sections were stored in 0.1M PBS. Floating sections were rinsed in 0.1% Triton X-100 (Fisher Scientific) in PBS, rinsed in PBS, and then blocked for 1 hr at room temperature in 8% normal goat serum (NGS, Jackson ImmunoResearch Laboratories) with 0.3% Triton X-100 in PBS. Sections were rinsed in PBS and for single labeling they were incubated overnight at 4° C. in 2% NGS, 0.3% Triton X-100 in PBS with primary antibody. The sections were then rinsed in PBS and incubated for 2 hr at room temperature with goat anti-rabbit IgG-FITC secondary antibody (1:1000, Millipore Bioscience International). Sections were rinsed again in PBS and mounted on slides using ProLong Gold antifade reagent with DAPI (Invitrogen). Primary antibodies used were HDAC3 (1:1000; Millipore Corporation), HDAC2 (1:1000; Abcam), HDAC4 (1:500; Abcam), and acetyl-histone-H4K8 primary antibody (1:1000; Cell Signaling Technology).

Images were acquired and using an Olympus (BX51, Japan) microscope using a 4× or 20× objective, CCD camera (QImaging), and QCapture Pro 6.0 software (QImaging) and quantified with ImageJ software (NIH). Primary antibodies used were HDAC3 (1:1000, Millipore), HDAC2 (1:1000, Abcam), HDAC4 (1:500, Abcam), and acetyl-Histone-H4K8 primary antibody (1:1000, Cell Signaling).

Quantitative Real-Time RT-PCR:

Quantitative real-time RT-PCR was performed to examine nuclear receptor subfamily 4 group A member 2(Nr4a2) and c-fos expression. Tissue was collected from 1 mm punches from dorsal hippocampal slices in the area of the focal deletion in HDAC3^{flox/flox} mice as confirmed by immunohistochemistry for HDAC3 and equivalent regions in HDAC3^{+/+} mice. RNA was isolated using RNeasy minikit (Qiagen, Carlsbad, Calif.). cDNA was made from 200 ng total RNA using the Transcriptor First Strand cDNA Synthesis kit (Roche Applied Sciences). Primers were derived from the Roche Universal ProbeLibrary: Nr4a2 left primer, 5′-ttgcagaatatgaacatcgaca-3′ [SEQ. ID NO. 1]; Nr4a2 right primer, 5′-gttccttgagcccgtgtct-3′ [SEQ. ID NO. 2]; Nr4a2 probe ttctcctg [SEQ. ID NO. 3]; c-Fos left primer 5′ ggggcaaagtagagcagcta 3′ [SEQ. ID NO. 4]; c-Fos right primer 5′ agctccctcctccgattc 3′ [SEQ. ID NO. 5]; c-Fos probe, atggctgc [SEQ. ID NO. 6], where both the Nr4a2 and c-Fos probes are conjugated to the dye FAM. Glyceraldehyde-3-phosphate dehydrogenase (GAPDH) left primer 5′ atggtgaaggtcggtgtga 3′ [SEQ. ID NO. 7]; right primer 5′ aatctccactttgccactgc 3′ [SEQ. ID NO. 8]; probe tggcggtattgg [SEQ. ID NO. 9], where the GAPDH probe is conjugated to Lightcycler Yellow 555. The non-overlapping dyes and quencher on the reference gene allow for multiplexing in the Roche LightCycle 480 II machine (Roche Applied Sciences). Analysis and statistics were performed using the Roche proprietary algorithms and REST 2009© software based on the Pfaffl method (Pfaffl, Nucleic Acids Res 29:e45, 2001; and Pfaffl et al., Nucleic Acids Res 30:e36, 2002, the disclosures of each of which are incorporated herein by reference.)

Object Recognition Protocol:

Training and testing for location-dependent object recognition memory (OLM) and novel object recognition memory (ORM) was carried out as previously described. (See, e.g., Roozendaal et al., J Neurosci 30:5037-5046, 2010, the disclosure of which is incorporated herein by reference.) Prior to training, mice were handled 1-2 min for 5 days and were habituated to the experimental apparatus 3 min a day for 3 consecutive days in the absence of objects. The experimental apparatus was a white rectangular open field (30×23×21.5 cm).

During the training trial, mice were placed in the experimental apparatus with two identical objects (either 100 ml beakers, 2.5 cm diameter, 4 cm height; or large blue Lego blocks, 2.5×2.5×5 cm) and were allowed to explore these objects for 3 min, which does not result in short- or long-term memory. (See, Stefanko et al., Proc Natl Acad Sci USA 106:9447-9452, 2009, the disclosures of which are incorporated herein by reference.) During the 24-h or 7-day retention test, mice were placed in the experimental apparatus for 5 min. For object recognition memory (ORM), one copy of the familiar object (A3) and a new object (B1) were placed in the same location as during the training trial. For location-dependent object recognition memory (OLM), one copy of the familiar object (A3) was placed in the same location as during the training trial and one copy of the familiar object (A4) was placed in the middle of the box.

All combinations and locations of objects were used in a balanced manner to reduce potential biases due to preference for particular locations or objects. All training and testing trials were videotaped and analyzed by individuals blind to the treatment condition and the genotype of subjects. A mouse was scored as exploring an object when its head was oriented toward the object within a distance of 1 cm or when the nose was touching the object. The relative exploration time was recorded and expressed by a discrimination index [DI=(tₙₒᵥₑₗ−t_{familiar})/(tₙₒᵥₑₗ+t_{familiar})×100].

Statistics:

Data sets with only two groups were analyzed by independent-samples t-test. Datasets with four groups, such as the HDAC3-FLOX and Nr4a2 siRNA experiment, were analyzed by two-way ANOVA, and separate one-way ANOVAs were used to make specific comparisons when significant interactions were observed. Student-Newman-Keuls and least significant different post hoc tests were performed when appropriate. Simple planned comparisons were made using Student's t tests with a levels held at 0.05.

### Example 1

### Generation of Focal HDAC3 Deletion

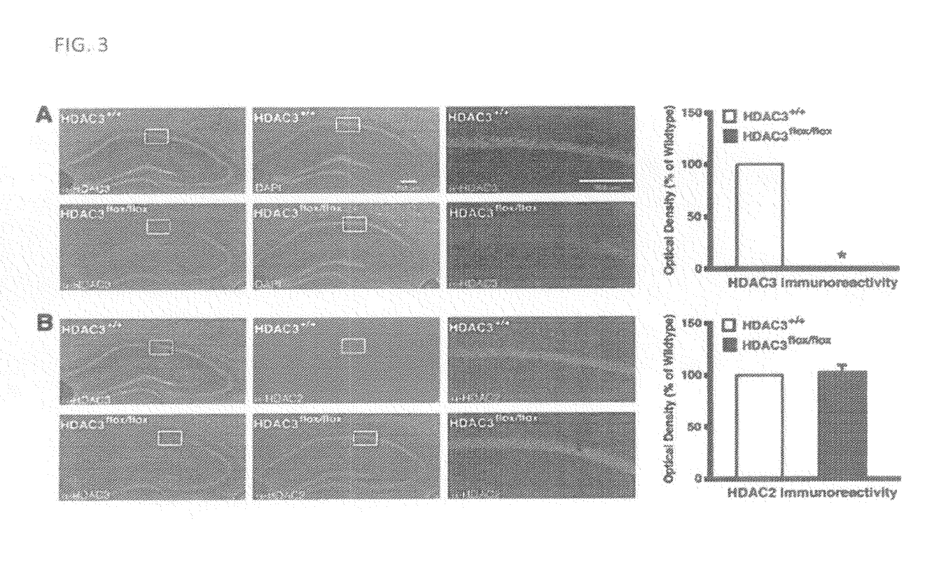

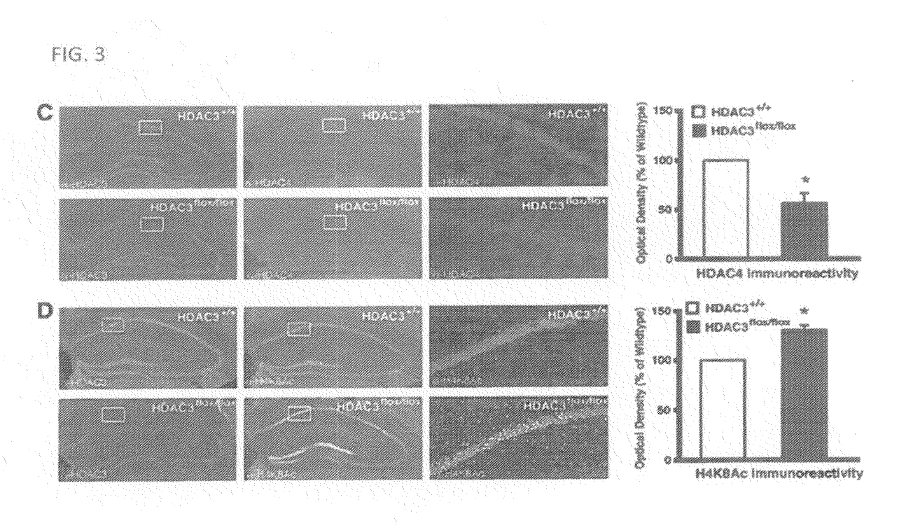

The overall goal of this study was to begin to understand the role of HDAC3 in long-term memory function. Focal deletions of HDAC3 allow for a detailed regional and task-selective behavioral analysis without developmental consequence. HDAC3^{flox/flox} and HDAC3^{+/+} mice received bilateral intrahippocampal infusions of AAV-Cre recombinase (1 μl/side) AAVserotype 2/1 was used, which has the viral genome of serotype 2 and packaged in coat proteins from serotype 1 for efficient transduction of dorsal hippocampal pyramidal neurons (Burger et al., 2004). This viral infusion does not alter neuronal morphology indicated by intact nuclei visualized by DAPI staining but does lead to a complete, focal deletion of HDAC3 as demonstrated by loss of immunoreactivity in the dorsal hippocampus (FIG. 3A, bottom left).

Next, immunoreactivity for HDAC2, another class I HDAC member, has been implicated in learning and memory (Guan et al., 2009, cited above), and it is part of a co-repressor complex with HDAC1 (Laherty C D, et al., Cell 89:349-356, 1993, the disclosure of which is incorporated herein by reference), and also HDAC4, a class IIa HDAC that can bind to HDAC3 in a co-repressor complex (Grozinger and Schreiber, 2000; and Fischle et al., 2002, cited above). HDAC3 deletion did not alter the expression of HDAC2 (FIG. 3B, bottom middle). In contrast, HDAC4 had reduced nuclear expression in the region of the HDAC3 deletion (F(1,6)=7.53; p=0.03) (FIG. 3C, bottom middle). These results suggest that deletion of HDAC3 has no observable effect, using immunohistochemical analysis, on expression of HDAC2; however, it has a significant effect on the expression of HDAC4.

To determine whether deletion of HDAC3 affected histone acetylation, acetylation of histone H4, lysine 8 (H4K8Ac) was examined. Acetylation at this site has been shown to increase after the dissociation of the NCoR/HDAC3 complex from promoter regions and consequently leads to an increase in transcriptional activity (Guenther et al., 2000 and Li et al., 2000, cited above; and Wang, D., et al., PLoS One, 5, e9853, 2010, the disclosure of which is incorporated herein by reference). Indeed, there was an observed increase in H4K8Ac in the region of HDAC3 deletion (F(1,5)=7.18; p=0.04) (FIG. 3D). These findings suggest that HDAC3, perhaps together with HDAC4, controls acetylation of H4K8 involved in transcriptional regulation (Agalioti T., et al., Cell 111:381-392, 2002 the disclosure of which is incorporated herein by reference).

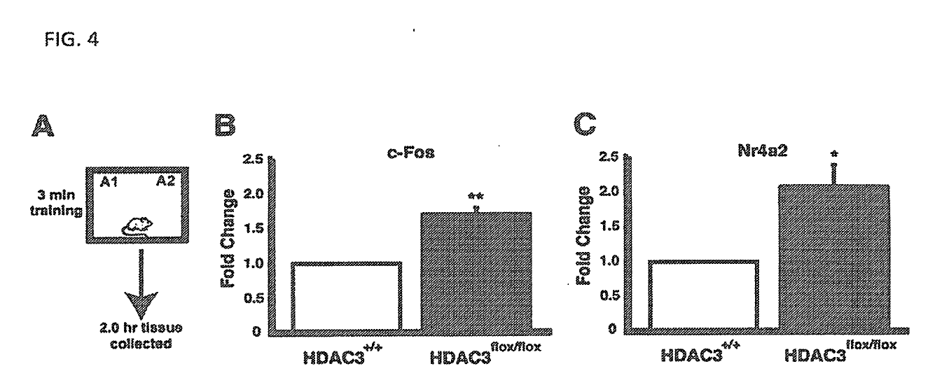

The absence of HDAC3, decreased expression of HDAC4, and the increase in histone acetylation suggested that gene expression would be increased in the region of the focal homozygous deletion of HDAC3. To test this, the expression of two immediate early genes, c-fos and Nr4a2, 2 h after object recognition training. Transcription of immediate early genes initiated by patterned synaptic activity is necessary for synaptic plasticity and long-term memory (for review, see Alberini, CM, Physiol Rev 89:121-145, 2009, the disclosure of which is incorporated herein by reference). It has been shown in a previous study that HDAC inhibition in the hippocampus maintained the expression of Nr4a2 at 2 h, beyond the point at which it would normally be expressed during memory consolidation (Vecsey et al., 2007, cited above). HDAC3^{flox/flox} and HDAC3^{+/+} mice received bilateral intrahippocampal AAV-Cre infusions 2 weeks (for optimal gene deletion; data not shown) before training. During training, mice were placed in an arena with two identical objects for a subthreshold 3 min training session (FIG. 4A), which does not result in long-term memory (Stefanko et al., 2009). Tissue was collected by taking 1 mm punches from dorsal hippocampal slices in the area of the focal deletion in HDAC3^{flox/flox} mice (n=3) as confirmed by immunohistochemistry for HDAC3 and equivalent regions in HDAC3^{+/+} mice (n=3).

C-fos expression was significantly increased in the area of the focal deletion of HDAC3^{flox/flox} mice compared with wild-type littermates (t(4)=6.81; p=0.002) (FIG. 4B). Similarly, Nr4a2 expression in the dorsal hippocampus was twofold greater in HDAC3^{flox/flox} compared with HDAC3^{+/+} mice after training (t(4)=4.05; p=0.015) (FIG. 4C). Gene expression was also measured in naive controls that received hippocampal AAV-Cre infusions to determine potential basal differences (data not shown). Naive handled HDAC3^{flox/flox} mice had significantly greater c-fos expression than wild-type mice (t(9)=2.30; p=0.05), yet basal Nr4a2 expression was unchanged (t(6)=0.33; p=0.75). Thus, Nr4a2 is differentially induced in the HDAC3^{flox/flox} mice, in which training triggers greater gene expression but basal levels are unchanged compared with HDAC3^{+/+} mice. Together, these data reveal that HDAC3^{flox/flox} mice have enhanced histone acetylation and gene expression in the focal deletion compared with wild-type controls.

### Example 2

### Effect of Deletion of HDAC3 in Dorsal Hippocampus

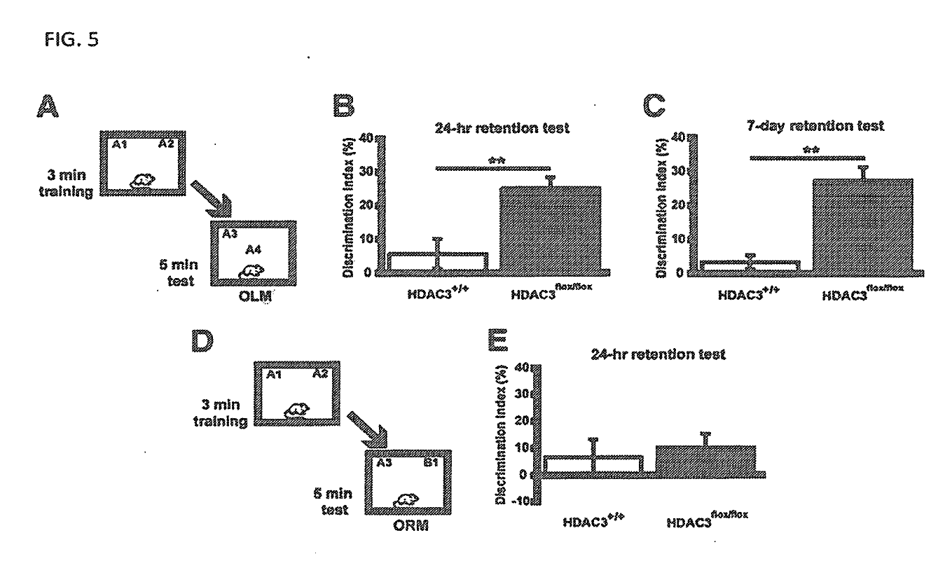

Previous studies have shown that HDAC inhibition enhances memory such that a subthreshold learning event that would not result in long-term memory is transformed into an event leading to long-term memory. (See, Stefanko et al., 2009, cited above.) To test if deletion of HDAC3 affects learning and memory in a similar manner, HDAC3^{flox/flox} and HDAC3^{+/+} mice received bilateral intrahippocampal AAV-Cre infusions two weeks (for optimal gene deletion and protein clearance) before training. During training, mice were placed in an arena with two identical objects for a 3-min training session, which does not result in long-term memory, and then tested 24 hours later in the same arena with one familiar object moved to a novel location (see FIG. 5A). (See, Stefanko et al., 2009, cited above.) Wildtype mice did not exhibit significant discrimination (n=8, DI=4.7±3.0%), confirming that 3 min was a subthreshold training period. In contrast, HDAC3^{flox/flox} mice displayed significant memory for object location, evident by a significantly greater discrimination index (n=8, DI=25.1±3.3%; t(14)=3.51; p=0.003; FIG. 5B). Groups did not differ in total exploration time of the two objects during either the training or retention test (data not shown). These results demonstrate that HDAC3 is a negative regulator of long-term memory in the dorsal hippocampus.

In the next experiment, the persistence of long-term memory induced by HDAC3 deletion was tested. Previously, it was demonstrated that novel object recognition after 10 min training is evident 24 hours later, but this memory fails when tested after 7 days (Stefanko et al. 2009, cited above). Mice received a 3 min training period followed 7 days later by a retention test. As shown in FIG. 5C, wildtype mice (n=9) did not exhibit long-term memory where as the HDAC3^{flox/flox} mice (n=9; DI=27.4±4.0%) did show significant long-term memory for object location (DI=27.4±4.0%; t(16)=5.30; p<0.001). Groups did not differ in total exploration time of the two objects during either the training or retention test (data not shown). These results suggest that HDAC3 deletion leads to long-term memory formation that is persistent and lasts beyond the point at which normal long-term memory fails.

Whether the focal HDAC3 deletion affected long-term memory in a standard novel object recognition task (ORM) was examined next. In this task, there is no change in context or object location, but one of the familiar objects is replaced with a novel object (see FIG. 5D). The dorsal hippocampus has been shown to encode information regarding context and location (O'Keefe J, Hippocampus 9:352-364, 1999; Fanselow M S, Behav Brain Res 110:73-81, 2000; Maren S & Holt W, Behav Brain Res 110:97-108, 2000; and Smith D M & Mizumori S J, J Neurosci 26:3154-3163, 2006, the disclosures of which are incorporated herein by reference); however, other brain regions, such as insular cortex, are important for long-term memory for the object itself (Balderas I, et al., Learn Mem 15:618-624, 2008; and Roozendaal B, et al., J Neurosci 30:5037-5046, 2010, the disclosures of which are incorporated herein by reference). This distinct neural circuitry for the ORM and OLM tasks can reveal the specificity of the treatment.

FIG. 5E shows that after subthreshold training (3 min), both HDAC3^{flox/flox} (n=8) and HDAC3^{+/+} mice (n=8) spent similar amounts of time with both the familiar and novel objects on test day (t(14)=0.40; p=0.70). Groups did not differ in total exploration time of the two objects during either the training or retention test (data not shown). Together, the data in FIGS. 4 and 5 suggest that HDAC3 deletion in the dorsal hippocampus results in a selective enhancement of long-term memory for the object location (FIG. 5B) but not the object itself (FIG. 5E).

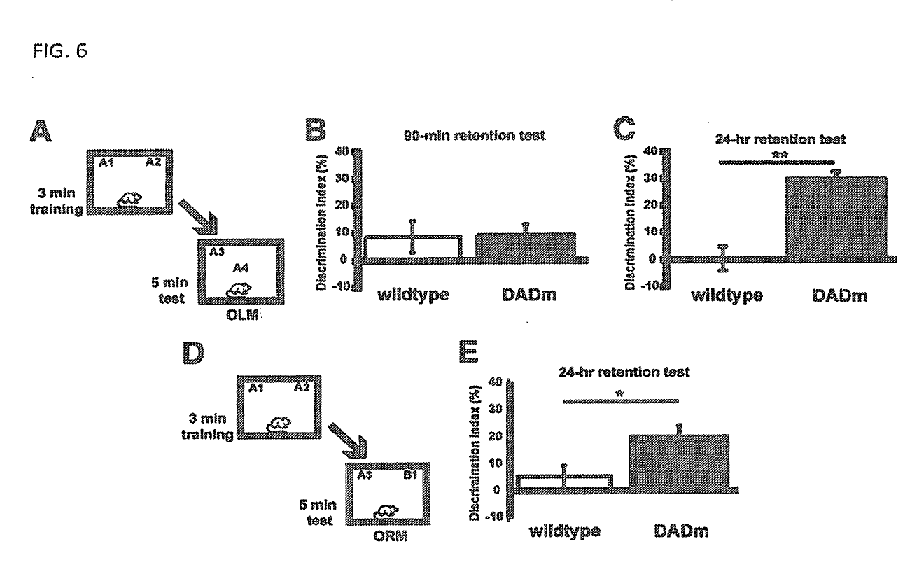

To further support the role of HDAC3 as a negative regulator of long-term memory formation, genetically modified NCoR homozygous knock-in mice were also examined. These mice (referred to as DADm mice) carry a single amino acid substitution (Y478A) in the deacetylase domain (DAD) of NCoR that disrupts its binding to HDAC3. (See, Alenghat et al., Nature 456:997-1000, 2008, the disclosure of which is incorporated herein by reference.) Mice were subjected to a subthreshold training period (3 min) and tested for short-term (90 min) memory for object location (FIG. 6A). As shown in FIG. 6B, DADm (n=11) and wildtype mice (n=13) performed similarly on a 90 min retention test (t(22)=0.08; p=0.94). In a different set of mice to examine long-term memory at 24 hrs (mice were also given a 3 min training period), DADm mice (n=10) exhibited significant memory for the location of the familiar object as compared to wildtype controls (n=9; t(17)=3.52; p=0.003) (FIG. 6C). Groups did not differ in total exploration time of the two objects during either the training or retention test (data not shown). Thus, disruption of the interaction between NCoR and HDAC3 is sufficient to disrupt HDAC3 function and capable of causing similar effects as HDAC inhibition by enhancing long-term, but not short-term, memory.

Next, the question of whether loss of NCoR/HDAC3 interactions affected long-term memory in the ORM task was examined. Because these are traditional knock-in mice, mutant NCoR is present in all cells expressing NCoR. Thus, it is predicted that DADm mice would exhibit enhanced memory in the ORM task (FIG. 6D) as well. FIG. 6E shows that, after subthreshold training (3 min), DADm mice (n=18) showed a greater preference for the novel object that the wild-type mice (n=12) on test day (t₍₂₈₎=2.19; p=0.04). These data suggest that brain regions mediating ORM, such as insular and perirhinal cortex, are also regulated by NCoR/HDAC3. Thus, disruption of the interaction between NCoR and HDAC3, which is sufficient to abrogate HDAC3 activity, results in similar effects as HDAC inhibition by enhancing long-term, but not short-term memory.

### Example 3

### RGFP136 Affect On HDAC3/HDAC4 Expression & Histone Acetylation

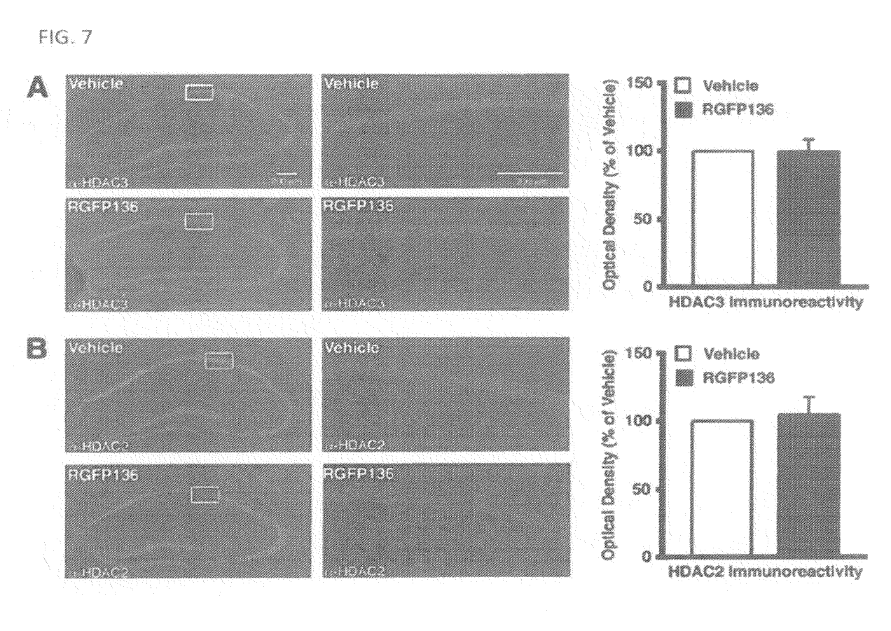

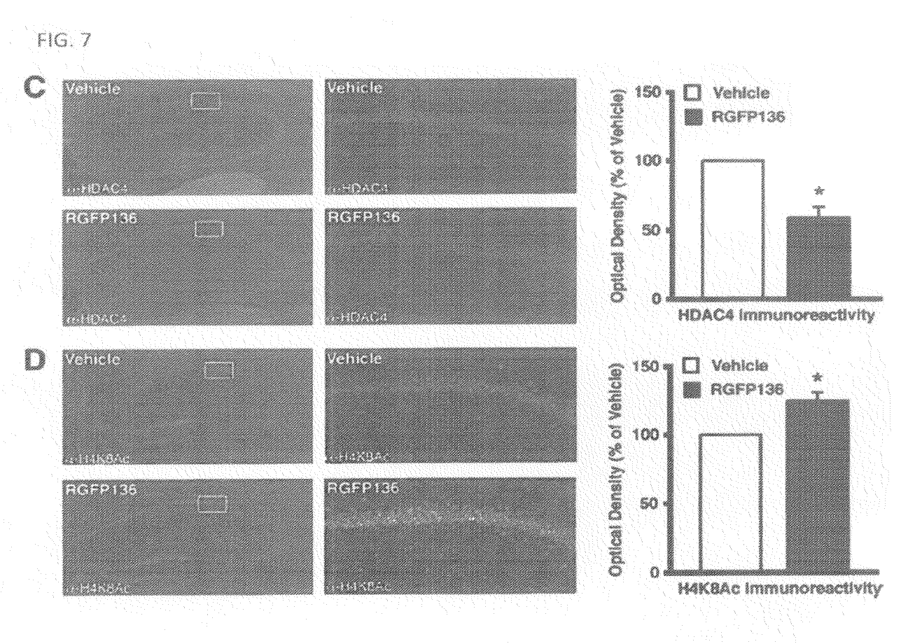

A new substituted or unsubstituted N-(o-aminophenyl)carboxamide HDAC inhibitor, RGFP136, has been characterized as a class I HDAC inhibitor with greatest inhibition of HDAC3 (Rai et al., 2010). This compound was then used to test whether acute inhibition of HDAC3 produced similar changes to that observed in the HDAC3^{flox/flox} mice with respect to HDAC2, 3, and 4 expression as well as histone acetylation. Brains from C57BL/6 mice with bilateral hippocampal cannulae were collected 2 hours after 0.5 μl infusions of RGFP136 (1.25 ng/side) or vehicle. The drug infusion does not alter neuronal morphology compared to vehicle as visualized by DAPI staining (data not shown). HDAC3 nuclear immunoreactivity is similar in drug-infused mice as compared to vehicle-treated mice (FIG. 7A, bottom right panel). HDAC2 expression is unchanged (FIG. 7B). Similar to results obtained from HDAC3^{flox/flox} mice shown in FIG. 3C, there is a loss of HDAC4 nuclear expression (F(1,5)=8.35, p=0.03; FIG. 6C).

To determine the effect of RGFP136 on histone acetylation, H4K8Ac was examined. Acetylation at this site has been shown to increase after the dissociation of the NCoR/HDAC3 complex from promoter regions and consequently leads to an increase in transcriptional activity (Wang et al., 2010). As predicted, RGFP136 infusion resulted in an increase of immunoreactivity for H4K8Ac compared to vehicle (F(1,6)=6.60, p=0.04; FIG. 7D). These findings mirror the results from HDAC3^{flox/flox} mice (FIG. 3) and further support that HDAC3 is responsible for HDAC4 nuclear localization and inhibition of HDAC3 results in increased histone acetylation.

### Example 4

### RGFP136 Treatment Effect on Long-Term Memory for Object Location

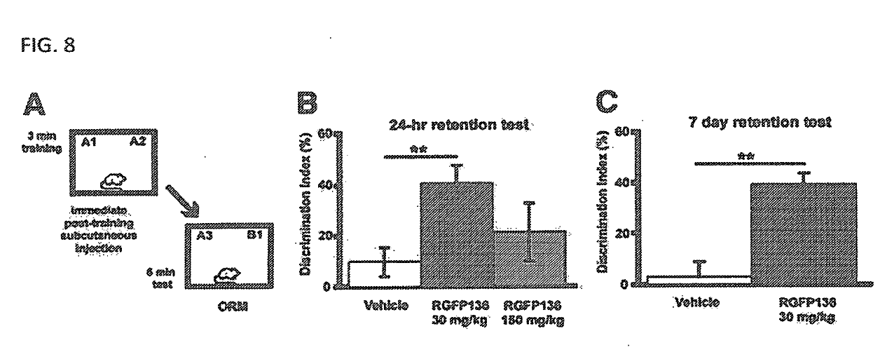

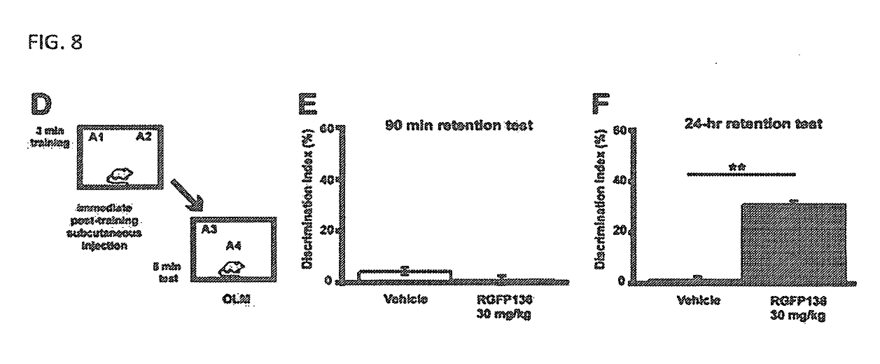

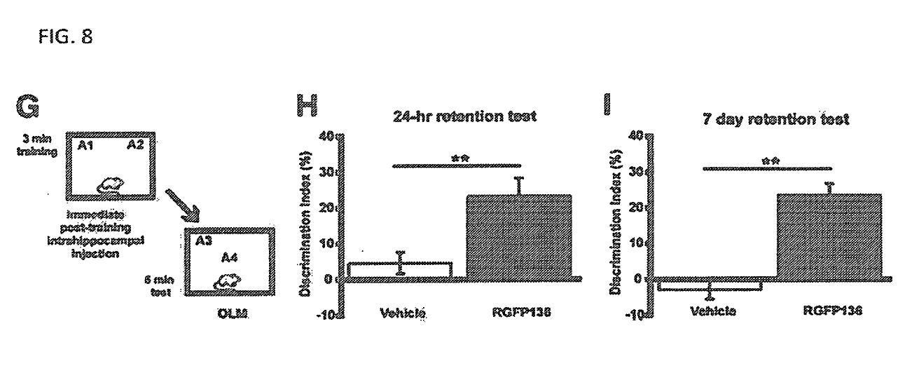

Next, the ability of RGFP136 to modulate long-term memory was examined. Mice were given a 3 min training period followed immediately by subcutaneous injection of RGFP136 (30 mg/kg or 150 mg/kg) or vehicle (FIG. 8A).

As shown in FIG. 8B, mice receiving the 30 mg/kg dose (n=9; DI=40.7±7.1%) exhibited significantly better long-term memory for the novel object than vehicle treated controls (n=7; DI=12.4±5.7%; one-way ANOVA F(1,14)=0.48, p=0.01). Since animals treated with 150 mg/kg RGFP136 did not exhibit significantly enhanced memory for the novel object (n=9, DI=21.5±11.5%; F(1,14)=4.83, p=0.530), only the low dose (30 mg/kg) was used to test the persistence of memory for the familiar object 7 days after the initial exposure in a different set of mice. Mice treated with 30 mg/kg RGFP136 immediately after a 3 min training period and tested 7 days later (n=10; DI=38.8±4.6) showed significant preference memory for the novel object (t(17)=2.06, p<0.05) as compared to vehicle controls (n=9; DI=2.5±6.3%; FIG. 8C). Groups did not differ in total exploration time of the two objects during either the training or retention test (data not shown).

Next, the effect of systemic delivery of RGFP136 (30 mg/kg, s.c.) for object location task was examined. Animals were tested for short-term memory 90 min after training. No significant preference for the object in the novel location was evident in either the RGFP136 or vehicle-treated groups (RGFP136, n=10, DI=0.5±1.4%; vehicle, n=9, DI=4.1±1.4%; t(17)=1.835; p<0.05) (FIG. 8E). In a different set of mice to examine long-term memory at 24 h (mice were also given a 3 min training period), mice treated with post-training RGFP136 (30 mg/kg, s.c.) exhibited significant preference for the object in the novel location compared with vehicle controls (F(1,17)=192.21; p±0.001) (FIG. 8F). These findings mirror the effects in HDAC3^{flox/flox} mice as well as a recent study using a general HDAC inhibitor (Stefanko et al., 2009, cited above), in which enhanced memory is demonstrated in long-term, but not short-term, memory tests.

To test the effect of site-specific delivery of RGFP136 on long-term memory, its ability to modulate memory for object location was examined (see FIG. 8G). Mice fitted with bilateral hippocampal cannulae received either 0.5 μl infusions of RGFP136 (n=7; 1.25 ng/side) or vehicle (n=7) immediately following a 3 min training period. Mice treated with RGFP136 showed greater memory 24 hours later for object location than controls treated with vehicle (t(12)=3.16; p=0.008; FIG. 8H). In addition, a separate group of mice were given a 3 min training period and then a 7 day retention test. RGFP136 treated mice (n=8) showed significantly greater memory for object location than vehicle treated controls (n=8; t(14)=6.10; p<0.001; FIG. 8I). Groups did not differ in total exploration time of the two objects during either the training or retention test (data not shown).

Next, whether intrahippocampal delivery of RGFP136 affected long-term memory in a standard novel object recognition task (ORM; data not shown) was examined. Both RGFP136 (DI=0.3±4.2%) and vehicle-treated mice (DI=0.9±6.7%) did not show a preference for the novel object 24 h later (p<0.05), demonstrating that site-specific delivery only enhances hippocampus dependent long-term memory.

RGFP136, used in these studies, has an IC50 of 3.0 nM for HDAC1, 2.1 nM for HDAC2, and 0.4 nM for HDAC3 using purified recombinant HDACs. Following systemic subcutaneous injection, the maximum drug concentration (Cmax) in the brain is approximately 1.7 μM for a 30 mg/kg dose. This suggests that following systemic administration, as in the data shown in FIG. 8, RGFP136 is at a sufficient concentration in the brain to inhibit HDAC3, but perhaps not HDAC1 or HDAC2. Further, the immunofluorescence data indicate that RGFP136 disrupts HDAC4 expression, with no effect on HDAC2 expression. Behaviorally, when delivered site-specifically to the dorsal hippocampus, RGFP136 transformed a learning event that does not result in long-term memory into an event that now does lead to long-term memory. Furthermore, this facilitation of long-term memory via RGFP136 resulted in persistent long-term memory observed 7 days later when normal long-term memory retrieval for object location fails. Subcutaneous injection of RGFP136 also facilitated long-term memory for object location (FIG. 8E) as well as long-term memory for a familiar object (FIG. 8B). These results collectively demonstrate that RGFP136 leads to similar effects on long-term memory for object location when delivered to the dorsal hippocampus as HDAC3 dorsal hippocampal deletion. Furthermore, these data reveal that RGFP136, a substituted or unsubstituted N-(o-aminophenyl)carboxamide HDAC inhibitor, modulates long-term memory formation.

### Example 5

### CBP Effect on RGFP136 Enhancement of Long-Term Memory

HDAC3 is found in the nucleus, cytoplasm, and plasma membrane where it can regulate transcription of genes as well as perform other nontranscriptional functions (e.g., deacetylate nonhistone proteins; reviewed in Karagianni and Wong, 2007). In order to test if the enhancements in memory formation observed in FIGS. 7 and 8 may be due to the transcription of genes necessary for long-term memory, genetically modified CREB-binding protein (CBP) mutant mice carrying a triple point mutation in the phospho-CREB (KIX) binding domain of CBP were studied. (For additional information on CBP^{KIX/KIX} mice, see, Kasper et al., Proc Natl Acad Sci USA 99:2320-2325, 2002, the disclosure of which are incorporated herein by reference.) Previously, it was demonstrated that HDAC inhibition, by either sodium butyrate or trichostatin A, enhances hippocampal synaptic plasticity via a CREB:CBP interaction (Vecsey et al., 2007, cited above). To see if RGFP136 is acting via a similar mechanism, this drug was infused into the dorsal hippocampus of CBP^{KIX/KIX} and CBP^{+/+} mice after a 3 min subthreshold training period and tested the effects on long-term memory.

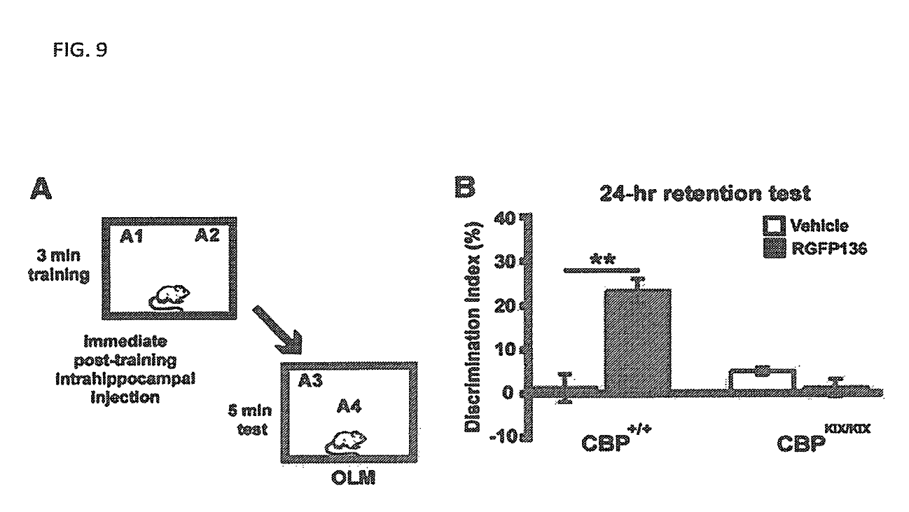

It was found that the overall effects of genotype [F(1,24)=17.12; p<0.001], drug treatment [F(1,24)=17.15; p<0.001], as well as interaction of genotype×drug treatment [F(1,24)=34.41; p<0.001]. RGFP136-treated CBP^{+/+} mice (n=5) showed significantly greater memory for novel object location than vehicle treated controls (n=6; p<0.001) (FIG. 9B). However, RGFP136 had no effect on long-term memory in the CBP^{KIX/KIX} mice (n=8 Vehicle, n=9 RGFP136; p=1.0; FIG. 9B). Groups did not differ in total exploration time of the two objects during either the training or retention test (data not shown). These results indicate that RGFP136 enhances memory through a CBP dependent mechanism.

Accordingly, it was found that in the hippocampus, RGFP136 requires CBP to facilitate long-term memory formation. CBP^{KIX/KIX} mice, which contain a mutation in the phospho-CREB (KIX) binding domain of CBP (Kasper et al., 2002, cited above), failed to exhibit significant long-term memory for object location when RGFP136 was delivered to the dorsal hippocampus. Although not to be bound by theory, these results suggest that RGFP136 is functioning via a CBP-dependent mechanism in order to regulate transcription required for long-term memory.

### Example 6

### RFGP Dose Response on Long-Term Memory for Object Recognition

C57BL/6J male mice were placed in the experimental apparatus with two identical objects and were allowed to explore these objects for 3 min, which does not result in short- or long-term memory (Stefanko et al., 2009, cited above). Immediately following training, mice received subcutaneous injections of either vehicle (20% glycerol, 20% PEG 400, 20% propylene glycol, and 100 mM sodium acetate, pH 5.4), RGFP 109 (3, 10, 30 mg/kg), RGFP 136 (3, 10, 30 mg/kg), RGFP 966 (3, 10, 30 mg/kg), or RGFP 999 (30 mg/kg). 24-h later mice were tested for memory retention (5 min) using the object recognition memory task, in which a familiar object was replaced with a novel one. A discrimination index (DI) above zero indicates a preference for the novel object.

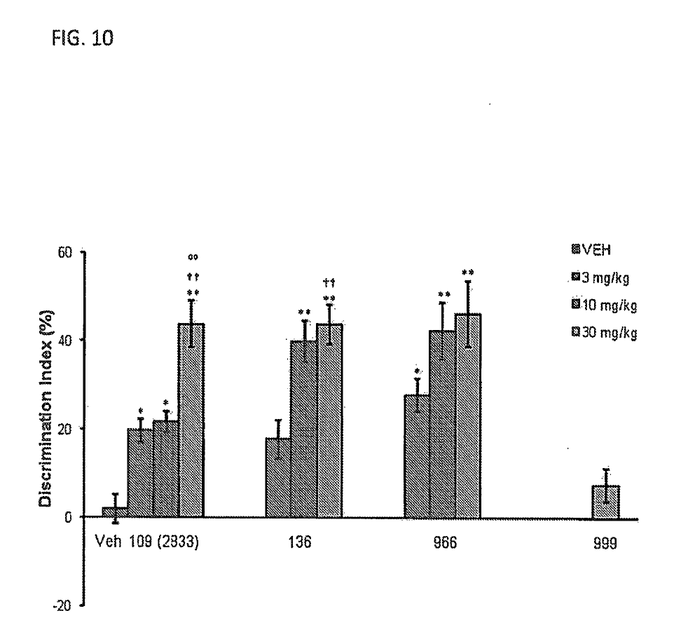

As shown in FIG. 10, doses of RGFP 109, 136, and 966 significantly enhanced long-term memory formation compared to vehicle-treated mice after subthreshold training [Dose: F(2,50)=14.61, p<0.001; Drug: F(3,50)=11.34, p<0.001]. RGFP 999, an inactive compound, did not demonstrate a significant preference as compared to vehicle-treated mice. Strong preferences for the novel object were formed by the highest dose of all active compounds (vs. Veh, ** p<0.001). Significant dose-dependent effects were seen with RGFP 109 and 136, but not for RGFP 966. The lack of an observed dose effect for RGFP 966 is likely due to its enhanced brain penetrance, and lower doses should be explored. While 3 mg/kg 966 dose group was not significant from the 30 mg/kg dose, it appeared to be on the threshold for producing a full behavioural effect. These data also provide the necessary information to identify the lowest behaviourally effective dose for future experiments.

### Example 7

### Deletion of HDAC3 Requires Nr4a2 Expression To Enhance Memory

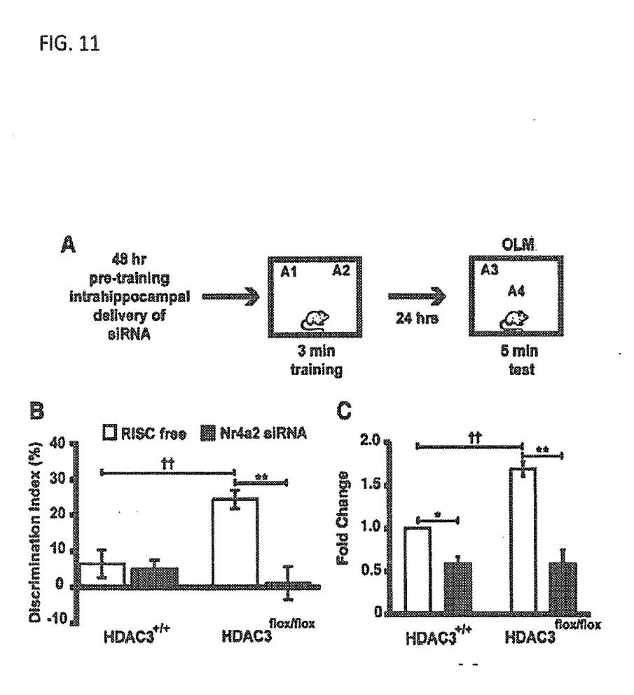

Nr4a2 is a CREB-dependent gene implicated in long-term memory (Pen~a de Ortiz S, et al., Neurobiol Learn Mem 74:161-178, 2000; von Hertzen L S & Giese K P, J Neurosci 25:1935-1942, 2005; Colo' n-Cesario W I, et al., Learn Mem 13:734-744, 2006; and Vecsey et al., 2007, cited above, the disclosures of which are incorporated herein by reference). In FIG. 4 it is shown that subthreshold training induced greater Nr4a2 gene expression in the dorsal hippocampi of HDAC3^{flox/flox} mice compared with wild-type controls. To determine whether this increase in Nr4a2 expression is necessary for enhanced long-term memory in HDAC3^{flox/flox} mice, siRNA targeting Nr4a2 was infused 48 h before training (FIG. 11A). An overall effect was found for siRNA treatment (F(1,35)=11.98; p=0.001), genotype (F(1,35)=5.08; p=0.03), and an interaction of genotype X siRNA treatment (F(1,35)=7.31; p=0.005). HDAC3^{flox/flox} mice infused with RISC-free siRNA (n=10) demonstrated significant preference for the object in the novel location, which was blocked by Nr4a2 siRNA treatment (n=10; p<0.001) (FIG. 11B). HDAC3^{+/+} mice did not display preference for the novel location after either RISC-free or Nr4a2 siRNA treatment (n=10 for RISC free and n=9 for Nr4a2 siRNA; p=1.0). Two hours after testing, brains were collected to determine levels of Nr4a2 mRNA in the dorsal hippocampus. Significant effects were found for genotype (F(1,8)=11.24; p=0.01), siRNA treatment (F(1,8)=56.45; p<0.001), and an interaction of genotype X siRNA treatment (F(1,8)=10.82; p=0.01). FIG. 11C shows that the infusion of Nr4a2 siRNA significantly decreased Nr4a2 expression in wild-type and HDAC3^{flox/flox} mice compared with RISC-free siRNA-infused controls (wild-type, p=0.02; HDAC3^{flox/flox}, p<0.001). In addition, HDAC3^{flox/flox} mice treated with RISC-free siRNA also demonstrated an increased induction of Nr4a2 mRNA after the long-term memory test (p=0.002 vs HDAC3^{+/+} RISC free). This enhancement posttest is similar to increases seen after training (FIG. 4B). As is discussed below, this data yield a potential mechanism for the negative regulation of long-term memory by HDAC3.

### Example 8

### Extinction and Reinstatement Experiments

Conditioned place preference experiments were similar to those described in Malvaez et al. (Malvaez, M., et al., Biological Psychiatry, 67, 36-43, 2010, the disclosure of which is incorporated herein by reference). Mice were briefly handled for 3 consecutive days (days 1-3). Baseline preferences were assessed by placing the animals in the three-chambered apparatus for 15 min (Pretest, day 4). Time spent in each compartment was recorded. Conditioning took place over the next 4 days with the guillotine doors closed, confining animals to a specific compartment for 30 min (days 5-8). An unbiased paradigm was used such that half of the animals were injected with cocaine (20 mg/kg, i.p.) before placement in the checkered compartment and half were injected with cocaine before placement in the white compartment (CS+). The next day, mice received 0.9% saline injection (1.0 ml/kg, i.p.) before placement in the alternate compartment (CS−). Injections were alternated for subsequent conditioning sessions. Forty-eight hours after the last conditioning session, animals had access to all 3 compartments and preference was assessed in a drug-free state (15 min, Posttest; day 10). This is also the first of the extinction sessions which occurred daily until extinction criteria were met. Immediately following this session, animals received an injection of either RGFP966 (3 or 10 mg/kg, s.c.) or vehicle alone (30% hydroxypropyl-β-cyclodextrin and 100 mM sodium acetate (pH 5.4); 1.0 ml/kg, s.c.) and were returned to their home cage. Animals continued extinction sessions on the following days with drug injections given only after Posttest and Ext2 (day 10 and 11). The a priori extinction criteria were defined as a preference for the cocaine-paired compartment (CS+) that is equal to or less than their initial preference as well as a no significant difference in time spent between the 2 compartments (CS+ vs. CS−).

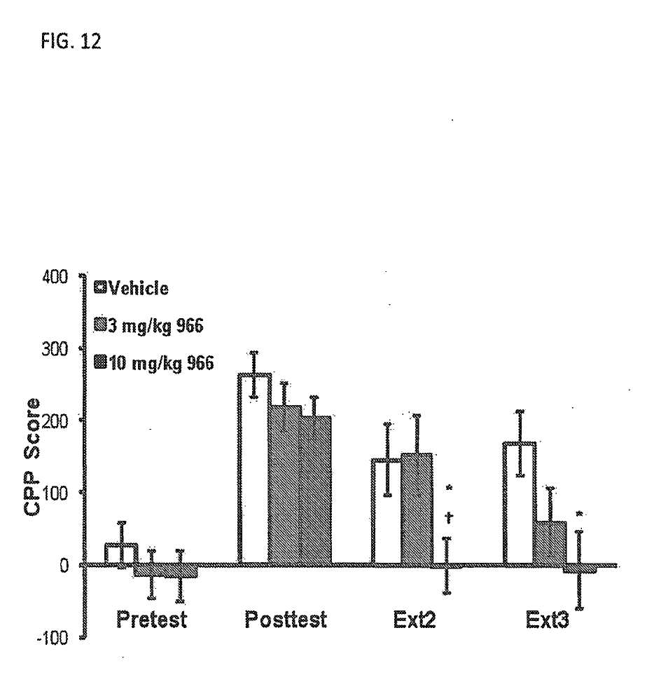

Data shown as a difference between time spent in the CS+ minus time spent in the CS− (CPP score indicates preference by mean±S.E.M. of time in CS+ minus CS-compartment) are provided in FIG. 12. All mice displayed a significant preference for the cocaine-paired compartment following conditioning (Posttest). Treatment with RGFP 966 (10 mg/kg, s.c.) immediately following Posttest resulted in rapid extinction of this preference as seen on the following extinction days (Ext2 and Ext3). *p<0.05 vs. Veh, †p<0.05 vs. 3 mg/kg 966, n=12/group.

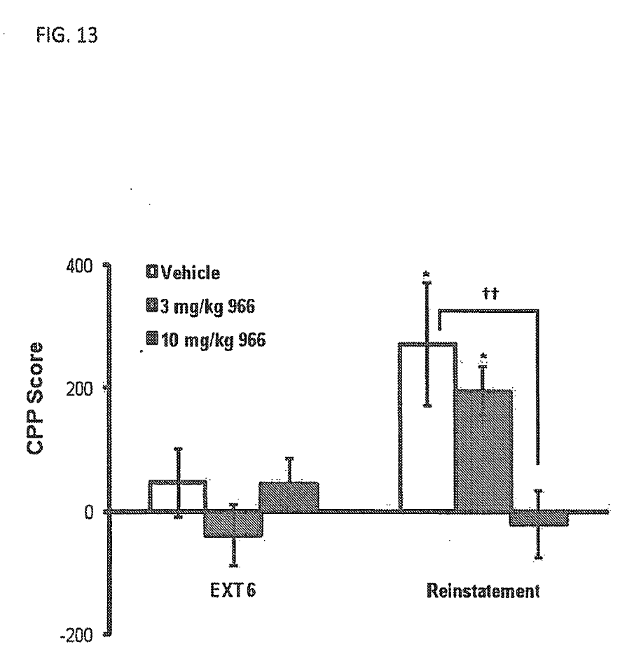

In an experiment with a similar design as described above, animals were confined to a specific compartment and given cocaine (5 mg/kg, i.p.) or saline on alternating days. Immediately following the conditioning session (30 min), mice received an injection of RGFP966 (10 mg/kg, s.c.) or vehicle and then returned to the home cage. Forty-eight hours after the last conditioning session, animals had free access to all 3 compartments and preference was assessed in a drug-free state (15 min, Posttest; day 10). CPP score indicates preference by mean±S.E.M. of time in CS+ minus CS− compartment. All groups extinguished the preference for the CS+ compartment by extinction day 6. The following day, animals received a cocaine prime (10 mg/kg, i.p.) and were placed in the test chamber (Reinstatement). Mice that had previously received vehicle and 3 mg/kg 966 showed a significant preference for the cocaine-paired compartment (paired t-test, * p<0.03 vs. Ext6), demonstrating that cocaine was able to reinstate cocaine-seeking behavior. However, mice that had previously received 10 mg/kg 966 during extinction did not reinstate a preference, thus extinction was refractive to reinstatement. †† p<0.01 vs. Vehicle, n=8/group, as shown in FIG. 13.

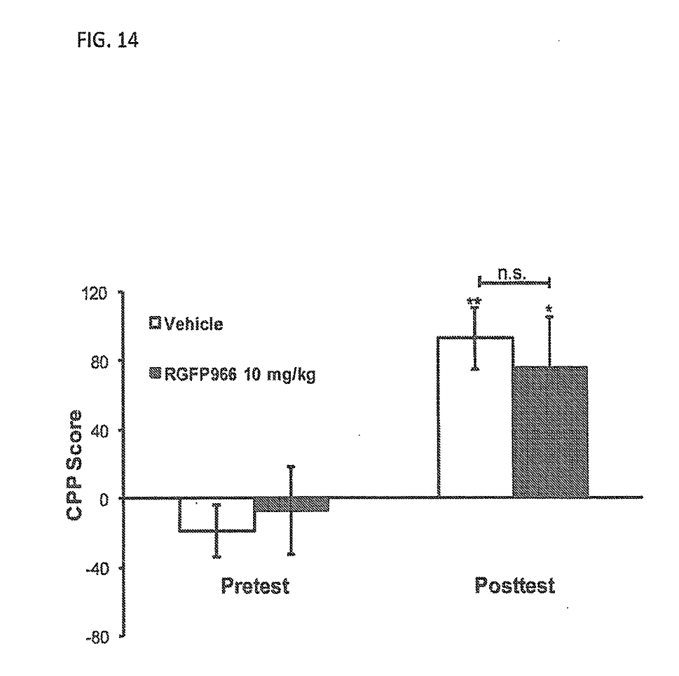

Finally, experiments were conducted that demonstrate that RGFP 966 treatment immediately following conditioning sessions does not alter cocaine conditioned place preference. In this test, mice were conditioned with two pairings of a low dose of cocaine (5 mg/kg, i.p.) and saline. Following each conditioning session, mice received RGFP966 (10 mg/kg, s.c.) or vehicle. All animals displayed a significant conditioned place preference (Posttest), and RGFP treatment did not alter this behavior. * p<0.02 **p<0.001 vs. pretest, n=14/group, as shown in FIG. 14.

### Example 9

### Study of Difference Between Function of ORM & OLM

A double dissociation between post-training sodium butyrate infusion into the insular cortex and dorsal hippocampus on the consolidation of memory for the familiar object (ORM task) itself and memory for object location (OLM task), respectively, has recently been observed. (Roozendaal et al., 2010, cited above.) The dorsal hippocampus has been shown to encode information regarding context and location; however other brain regions, such as insular cortex, are important for long-term memory for the object itself. (See, e.g., O'Keefe, Hippocampus 9:352-364, 1999; Fanselow, Behav Brain Res 110:73-81, 2000; Maren & Holt, Behav Brain Res 110:97-108, 2000; Smith & Mizumori, J Neurosci 26:3154-3163, 2006; Balderas et al., 2008; and Roozendaal et al., 2010, the disclosures of each of which are incorporated herein by reference.) This distinct neural circuitry for the ORM and OLM tasks reveals the specificity of the inventive treatment. Focal deletion of HDAC3 in the dorsal hippocampus resulted in a selective enhancement of OLM formation, but no effect on ORM (FIG. 5). Hippocampal infusion of RGFP136 also enhanced long-term OLM formation (FIG. 8). Homozygous NCoR knockin mice (DADm mice; FIG. 6), which have reduced HDAC3 activity throughout the brain, and systemic injections of RGFP136 (FIG. 8) resulted in enhancement of both OLM and ORM. These results combined indicated that the methodology of the instant invention can be used to enhance and regulate both OLM and ORM.

### SUMMARY

These experiments demonstrate the role of HDAC3 as a critical negative regulator of long-term memory formation. A number of important discoveries have been made that prove the efficacy of down-regulating HDAC3/4 as a therapeutic method of regulating long-term memory and treating memory dysfunction, including:
Focal homozygous gene deletion of HDAC3 resulted in not only loss of HDAC3, but also a significant decrease in HDAC4 nuclear expression.
Neurons lacking HDAC3 had increased histone acetylation of histone H4 lysine 8 (H4K8Ac), which correlated with increased c-fos and Nr4a2 expression in the area of the focal HDAC3 deletion in the dorsal hippocampus.
Focal homozygous deletion of HDAC3 in the dorsal hippocampus lead to facilitated long-term memory formation after a subthreshold training period. This subthreshold training period failed to yield long-term memory in control animals.
HDAC3 may modulate long-term memory formation via the expression of the immediate early gene and transcription factor Nr4a2, providing a specific target for inhibitor and gene therapies.

The genetic approach to examine the role of HDAC3 in long-term memory formation was complemented with a pharmacological approach using HDAC inhibitors from the substituted or unsubstituted N-(o-aminophenyl)carboxamides family, such as, for example, RGFP136, 109 and 966, which have been shown to be more selective for HDAC3 than other class I HDACs. These compounds, when delivered to the dorsal hippocampus resulted in decreased HDAC4 expression, increased H4K8Ac, and also significantly facilitated long-term memory formation. Further, these selective inhibitors facilitated long-term memory formation via a CBP-dependent manner in the hippocampus. Together, these genetic and neuropharmacological approaches identify HDAC3 as a critical negative regulator of memory.

To complement the genetic and pharmacological approach to study HDAC3, genetically modified NCoR mutant mice were also used. These mice, referred to as DADm mice, carry a single amino acid substitution (Y478A) in the NCoR deacetylase activation domain (DAD) of NCoR, which results in a mutant NCoR protein that is unable to associate with or activate HDAC3 (Ishizuka T & Lazar M A, Mol Endocrinol 19:1443-1451, 2005; and Guenther et al., 2001 and Alenghat et al., 2008, cited above, the disclosures of which are incorporated herein by reference). When given a subthreshold training period, DADm homozygous knockin mice exhibited significant long-term memory as compared to wildtype littermates, which failed to show any long-term memory. These data support the idea that a function complex between NCoR and HDAC3 is required to repress long-term memory formation.

Another major finding in this study is the relationship of hippocampal HDAC3 deletion with increased Nr4a2 expression. Nr4a2 is a CREB-dependent gene that has been implicated in long-term memory (Pen~a de Ortiz et al., 2000; von Hertzen and Giese, 2005; Colo' n-Cesario et al., 2006; and Vecsey et al., 2007, cited above). It has been demonstrated that Nr4a2 expression is enhanced by the HDAC inhibitor TSA during memory consolidation (Vecsey et al., 2007, cited above). It has now been discovered that enhanced Nr4a2 expression in HDAC3^{flox/flox} mice after learning (FIG. 4C). It has been suggested that HDACs may terminate the CREB-dependent transcription for this gene (Fass D M, et al., J Biol Chem 278:43014-43019, 2003, the disclosure of which is incorporated herein by reference), and thus the removal of HDAC3 allows transcription to be maintained for a longer period. Accordingly, it has been shown that activation of Nr4a2 is critical for the expression of long-term memory, as demonstrated by the current behavioral study using siRNA (FIG. 11). For example, HDAC3^{flox/flox} mice with a homozygous deletion of HDAC3 in the dorsal hippocampus failed to exhibit enhanced long-term memory when Nr4a2 siRNA was infused into the area of HDAC3 deletion before training. This data suggests a mechanism by which the loss of HDAC3 enhances long-term memory by allowing increased and/or prolonged CREB/CBP-dependent transcription of Nr4a2.

In summary, the current invention demonstrates that HDAC3 is a critical negative regulator of long-term memory formation. RGFP136, a substituted or unsubstituted N-(o-aminophenyl)carboxamide compound, represents a promising pharmacotherapeutic approach for cognitive impairments. Selective inhibitors and genetic manipulation of HDAC3 (via HDAC3^{flox/flox} and DADm mice) had similar effects at the molecular and behavioral level. Although not to be bound by theory it is likely that HDAC3 carries out its role in memory processes via its interactions with NCoR as well as HDAC4. Accordingly, gene therapies targeting the HDAC3/4/NCor complex may also be used to down-regulate HDAC3/4 and thereby also lead to regulation of long-term memory formation and treatment of memory dysfunction.

### DOCTRINE OF EQUIVALENTS

Those skilled in the art will appreciate that the foregoing examples and descriptions of various preferred embodiments of the present invention are merely illustrative of the invention as a whole, and that variations of the present invention may be made within the spirit and scope of the invention. For example, it will be clear to one skilled in the art that alternative dosing techniques or alternative treatment methodologies would not affect the overall HDAC3/4 specific memory regulation therapy of the current invention nor render it unsuitable for its intended purpose. Accordingly, the present invention is not limited to the specific embodiments described herein but, rather, is defined by the scope of the appended claims.

What is claimed is:

1. A method of regulating the transcription required for long-term memory formation for treating a memory disorder comprising:
administering a therapeutic amount of a pharmaceutical that selectively down-regulates the functional activity of at least one of HDAC3 and HDAC4 to a patient diagnosed with the memory disorder.

2. The method of claim 1, wherein the pharmaceutical at least comprises a therapeutically effective amount of a substituted or unsubstituted N-(o-aminophenyl)carboxamide that selectively inhibits at least one of HDAC3 and HDAC4.

3. The method of claim 1, wherein the pharmaceutical at least comprises a molecule selected from the group consisting of RGFP136, RGFP 109 and RGFP966.

4. The method of claim 1, wherein the pharmaceutical is delivered one of either systematically or intrahippocampally.

5. The method of claim 1, wherein the pharmaceutical enhances synaptic plasticity via a CREB:CBP interaction.

6. The method of claim 1, wherein the pharmaceutical induces greater gene expression in at least one of c-fos and Nr4a2.

7. The method of claim 1, wherein the memory disorder is selected from the group consisting of cognitive disorders, neurodegenerative diseases, and aging.

8. The method of claim 1, wherein the memory disorder is a memory that requires extinction.

9. The method of claim 8, wherein the memory that requires extinction relates to a memory selected from the group consisting of drug addiction and post traumatic stress.

10. The method of claim 8, wherein the pharmaceutical further prevents reinstatement of the memory.

11. The method of claim 1, wherein the pharmaceutical down-regulates the functional activity of both HDAC3 and HDAC4.

12. The method of claim 1, wherein the pharmaceutical enhances both object recognition and object location long-term memory.

13. The method of claim 1, wherein the pharmaceutical increases acetylation of H4K8.

14. A pharmaceutical compound for the treatment of a memory disorder comprising:
a therapeutically effective amount of at least one medicament that selectively down-regulates the functional activity of at least one of HDAC3 and HDAC4.

15. The compound of claim 14, wherein the pharmaceutical at least comprises a therapeutically effective amount of a substituted or unsubstituted N-(o-aminophenyl)carboxamide compound that selectively inhibits at least one of HDAC3 and HDAC4.

16. The compound of claim 14, wherein the pharmaceutical at least comprises a molecule selected from the group consisting of RGFP136, RGFP 109 and RGFP966.

17. The compound of claim 14, wherein the pharmaceutical is delivered one of either systematically or intrahippocampally.

18. The compound of claim 14, wherein the pharmaceutical enhances synaptic plasticity via a CREB:CBP interaction.

19. The compound of claim 14, wherein the pharmaceutical induces greater gene expression in at least one of c-fos and Nr4a2.

20. The compound of claim 14, wherein the memory disorder is selected from the group consisting of cognitive disorders, neurodegenerative diseases, and aging.

21. The compound of claim 14, wherein the memory disorder is a memory that requires extinction.

22. The compound of claim 21, wherein the memory that requires extinction relates to a memory selected from the group consisting of drug addiction and post traumatic stress.

23. The compound of claim 21, wherein the pharmaceutical further prevents reinstatement of the memory.

24. The compound of claim 14, wherein the pharmaceutical down-regulates the functional activity of both HDAC3 and HDAC4.

25. The compound of claim 14, wherein the pharmaceutical enhances both object recognition and object location long-term memory.

26. The compound of claim 14, wherein the pharmaceutical increases acetylation of H4K8.

27. A method of regulating the transcription required for long-term memory formation for treating a memory disorder comprising:
inserting a point mutation by a gene therapy technique to disrupt the NCoR/HDAC3/HDAC4 complex to selectively down-regulate the functional activity of at least one of HDAC3 and HDAC4 in a patient diagnosed with the memory disorder.

28. The method of claim 27, wherein the point mutation enhances synaptic plasticity via a CREB:CBP interaction.

29. The method of claim 27, wherein the point mutation induces greater gene expression in at least one of c-fos and Nr4a2.

30. The method of claim 27, wherein point mutation is introduced via AAV-Cre infusions.

31. The method of claim 27, wherein the point mutation is introduced into the dorsal hippocampus.

32. The method of claim 27, wherein the memory disorder is selected from the group consisting of cognitive disorders, neurodegenerative diseases, and aging.

33. The method of claim 27, wherein the memory disorder is a memory that requires extinction.

34. The method of claim 33, wherein the memory that requires extinction relates to a memory selected from the group consisting of drug addiction and post traumatic stress.

35. The method of claim 33, wherein the gene therapy further prevents reinstatement of the memory.

36. The method of claim 27, wherein the gene therapy down-regulates the functional activity of both HDAC3 and HDAC4.
